(19) United States
(12) Patent Application Publication
(10) Pub. No.: US 2024/0156403 A1
(43) Pub. Date: May 16, 2024

(54) Mind Balance Training (MBT) Platform

(71) Applicant: Northeastern University, Boston, MA (US)

(72) Inventors: Susan Whitfield-Gabrieli, Cambridge, MA (US); Clemens Bauer, Roslindale, MA (US); Jiahe Zhang, Boston, MA (US)

(21) Appl. No.: 18/502,948

(22) Filed: Nov. 6, 2023

Related U.S. Application Data
(60) Provisional application No. 63/382,720, filed on Nov. 7, 2022.

(51) Int. Cl. A61B 5/00; A61M 21/02; G01R 33/48

(52) U.S. Cl. CPC A61B 5/486; A61M 21/02; G01R 33/4806; A61B 5/742; A61M 2230/10

(57) ABSTRACT

A neurofeedback method and associated system for modulating brain activity includes functionally locating at least two brain networks in an adolescent subject which are associated with affective disorder symptoms during adolescence. Signals are recorded from each of the brain networks while the subject is performing a mediation task. An activity metric is determined based on a difference derived from the recorded signals, wherein changes in the activity metric over time are indicative of changes in relative activity level of the brain networks. Neurofeedback is provided by delivering a representation of the activity metric to the subject during the recording, thereby modulating brain activity in the subject.

### RELATED APPLICATION

This application claims the benefit of U.S. Provisional Application No. 63/382,720, filed on Nov. 7, 2022. The entire teachings of the above application are incorporated herein by reference.

### GOVERNMENT SUPPORT

This invention was made with government support under Grant No. MH113751-01A1 from the National Institutes of Health. The government has certain rights in the invention.

### BACKGROUND

Mental illness affects one in five adults in the United States and nearly 50 percent of all adolescents. [1] Large cohort studies have shown a majority of patients are initially prescribed ineffective pharmacological treatments, and it frequently takes years of trial and error to get to an effective therapy. [2, 3] While pharmacological interventions have improved the quality of life for a vast number of individuals, many drugs still cause serious side effects, and many are expensive, particularly when prescribed indefinitely. Behavioral therapies, such as mindfulness, have also been demonstrated to alleviate symptoms of many psychiatric disorders, and can be practiced without the burden of off-target effects or cost. [4] However, unlike oral drugs, behavioral therapies require time and focus of the practitioner; and, as currently implemented, these interventions are effective for only about 50% of patients. [5]

Additionally, adolescents experience alarmingly high rates of major depressive disorder (MDD); however, gold-standard treatments are only effective for a fraction of the youth. Accordingly, there is a critical need to develop novel interactions, particularly ones that target neural mechanisms believed to potentiate depressive symptoms.

### SUMMARY

A neurofeedback method for modulating brain activity includes functionally locating at least two brain networks in an adolescent subject during a resting state, at least one of the brain networks being associated with affective disorder symptoms during adolescence. Signals are recorded from each of the brain networks while the subject is performing a meditation task. An activity metric is determined based on a difference derived from the recorded signals, where changes in the activity metric over time are indicative of changes in relative activity level of the brain networks. Neurofeedback is provided by delivering a representation of the activity metric to the subject during the recording, thereby modulating brain activity in the subject.

The method can include functionally locating by using functional magnetic resonance imaging to generate a network map for each of the brain networks.

The signals can be blood-oxygen-level-dependent (BOLD) signals recorded using real-time functional magnetic resonance imaging (rt-fMRI).

The signals can be electrical signals recorded using fMRI compatible simultaneous electroencephalography.

The method can modulate the brain activity by downregulating, or decreasing activity level in one of the brain networks.

The method can modulate the brain activity by upregulating, or increasing activity level in one of the brain networks.

The meditation task of the method may include mindfulness meditation.

The brain networks of the method can include a default mode network (DMN), a central executive network (CEN), or a frontoparietal control network (FPCN).

The method may further include determining a first resting state activity before the performing of the meditation task, determining a second resting state activity after the performing of the meditation task, and assessing a change in functional connectivity of at least one of the brain networks based on a comparison between the first and second resting state activities.

The method may include the subject suffering from a major depressive disorder.

A neurofeedback system for modulating brain activity includes a processor, a sensor, and a memory with computer code instructions stored thereon, processor, the sensor, and the memory being configured to cause the system to functionally locate at least two brain networks in an adolescent subject during a resting state, the at least one brain networks being associated with affective disorder symptoms during adolescence. The system includes recoding signals from each of the brain networks while the subject is performing a meditation task. The system further includes determining an activity metric based on a difference derived from the recorded signals, wherein changes in the activity metric over time are indicative of changes in relative activity level of the brain networks. The system also provides neurofeedback to the subject by delivering a representation of the activity metric to the subject during the recording, thereby modulating brain activity in the subject.

The system will functionally locate the brain networks by using frequency specific components of electroencephalography signals as correlates of functional magnetic resonance imaging to generate a network map for each of the brain networks.

In the system, the electroencephalography signals are recorded on the scalp of the user wearing the system.

In the system, the using frequency specific components of the electroencephalography signals as correlates of functional magnetic resonance imagine further includes using user specific electroencephalography signals processed with user specific functional magnetic resonance imaging data to generate central executive network (CEN) dynamics, default mode network (DMN) dynamics, or both.

In the system, the modulating may include downregulating or decreasing activity level in one of the brain networks.

In the system, the modulating may include upregulating or increasing activity level in one of the brain networks.

In the system, the meditation task may include mindfulness meditation.

In the system, the brain networks include a default mode network (DMN), a central executive network (CEN), or a frontoparietal control network (FPCN).

The system may further include determining a first resting state activity level before the performing of the meditation task, determining a second resting state activity level after the performing of the meditation task, and assessing a change in functional connectivity of at least one of the brain networks based on a comparison between the first and second resting state activity levels.

The neurofeedback in the system may be provided visually on a screen.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in drawings in the manuscript being filed herewith. The drawings are not necessarily to scale, emphasis instead being placed upon illustrating embodiments.

FIG. 1 shows a flowchart of a method for modulating brain activity according to an example embodiment.

FIG. 2 shows a system flow for providing neurofeedback to a client in a chaperoned setting according to an example embodiment.

FIG. 3 shows a system flow for providing neurofeedback to a client in a relaxed (i.e., at home) setting.

FIGS. 4A-4B illustrate visualization of brain activity during real time fMRI neurofeedback training according to an example embodiment. In each figure, the left panel shows the experimenter view and the right panel show the subject view.

FIG. 5 shows an embodiment where a “game” of moving the white dot is used as a visual representation of mindfulness.

FIG. 6 is an example workflow relating to the expectations and outcomes of various steps of an example embodiment.

FIG. 7 is a block diagram of a neurofeedback system that may be used to modulate brain activity in an adolescent subject according to an example embodiment.

FIGS. 8A-8C illustrate the results of an experiment showing that higher DMN functional connectivity was associated with more severe depression symptoms at baseline.

FIGS. 9A-9C illustrate the results of an experiment where one session of mbNF reduced DMN functional connectivity.

FIGS. 10A-10B illustrate the results of an experiment where one session of mindfulness-based fMRI (functional magnetic resonance imaging) neurofeedback (mbNF) induced state mindfulness change.

### DETAILED DESCRIPTION

A description of example embodiments follows.

Imagine a future where children could get help before they are in mental crisis, through early detection of vulnerable brain states; and where children in mental distress could get treated efficiently and effectively with novel, precision, non-invasive therapies. To realize this vision, embodiments of the present invention aim to bridge the gaps in our understanding of the connections between neural network activity patterns, mental states, clinical diagnoses, and behavioral interventions on an individualized level. Embodiments disclose a Mind Balance Training platform; and it will provide targeted, low cost, effective, safe and equitable therapies, for multiple categories of patients with psychiatric symptoms. Methods and systems strive to create solutions for patients who are in need, some of whom are acutely suffering, with therapeutic approaches that are both individualized and scale-able across large populations.

Embodiments disclosed herein address the need for more effective, safe, and equitable therapies which are urgently needed for people suffering from psychiatric disorders. Real-time functional magnetic resonance imaging (rt-fMRI) presents an exciting opportunity to close the large gap between trial and error-based prescription practices, and true precision therapies. This is due to a growing recognition of neuropsychiatric conditions as disorders of functional interactions within and between brain signaling networks, which can be measured by rt-fMRI on an individualized basis. [6, 7, 8, 9] In particular, numerous fMRI studies show that in healthy adults, the brain's default mode network (DMN) shows an inverse relationship (or “anticorrelation”) with other large-scale networks, including for example, the frontoparietal control network (FPCN). [10] However, clinical therapies which specifically target these networks to alleviate psychiatric symptoms have not been developed. Embodiments propose an approach that incorporates fMRI-based neural “signatures” of brain states, along with conventional psychiatric diagnoses, to systematically develop effective, safe, equitable and highly individualized therapies.

Adolescents experience alarmingly high rates of major depressive disorder (MDD); however, gold-standard treatments are only effective for a fraction of the youth. By directly addressing this gap, embodiments illustrate a mindfulness-based fMRI (functional magnetic resonance imaging) neurofeedback (mbNF) for adolescents that aims to reduce default mode network (DMN) hyperconnectivity, which has been implicated in the onset and maintenance of MDDs. Embodiments provide a Mind Balance Training Platform system and method for the development of precision behavioral therapies. Embodiments will sequentially: (i) bring together individuals and medical practitioners to implement therapies in a technology intensive clinical setting; (ii) enable continued training in a naturalistic (i.e., at-home) environment with less intensive technology-assistance, and ultimately; (iii) prepare individuals to incorporate precision behavioral interventions into everyday life without the use of technology. This platform is a novel, precision-psychiatry method that helps people normalize their dysfunctional neural interactions. These systems and methods have the potential to improve mental wellness among individuals afflicted with a variety of affective disorders, as well as children at-risk for developing mental illness.

As used herein, the term “affective disorder” refers to any of several psychological disorders characterized by abnormalities of emotional state and including especially major depressive disorder, dysthymia, and bipolar disorder. Types of affective disorders include but are not limited to, for example, Unipolar Depression and its variants including Postpartum Depression, Atypical Depression, and Seasonal Affective Disorder (SAD); Bipolar Disorder; Dysthymia and Cyclothymia; Generalized Anxiety Disorder; Panic Disorder; Phobias including Agoraphobia; Obsessive Compulsive Disorder (OCD); and Post-traumatic Stress Disorder (PTSD). Affective disorder, as used herein, may also refer to mental conditions such as, but not limiting to, attention deficit hyperactive disorder (ADHD) as well as schizophrenic hallucinations.

It has been shown that DMN-FPCN (default mode network—frontoparietal control network) network dynamics characterize attention deficit hyperactive disorder (ADHD) symptoms and predict ADHD onset in vulnerable populations. While brain imaging is not currently used as a diagnostic tool, major neural circuits hypothesized to underlie various ADHD symptoms (i.e., inattention) have been well described using fMRI approaches. Inattention and executive function deficits are associated with decreased DMN-FPCN anticorrelations in healthy controls [9] as well as in populations with cognitive impairments (e.g., individuals with ADHD). [22] Moreover a brain network-based marker of mind wandering has been identified, a symptom associated with ADHD. [23] DMN-FPCN connectivity also predicts worsening of ADHD symptoms years later in young children with no current clinical diagnosis. [24]

Further, ADHD symptoms are susceptible to pharmaceutical and behavioral interventions in neuropsychiatric populations. In two separate studies, pharmacological and behavioral interventions have been employed to increase DMN-FPCN anticorrelations which resulted in an associated increase in cognitive performance. [13, 25] For example, in sixth grade school children, mindfulness meditation interventions increased sustained attention, which was also associated with increased DMN-FPCN anticorrelations. [26] Thus, it is shown that DMN-FPCN anticorrelation characterizes ADHD and predicts ADHD in some individuals. But therapies designed to directly target these networks in ADHD patients are lacking. In various non-ADHD clinical populations, it has been previously demonstrated that mindfulness reduces ADHD-associated symptoms such as mind wandering, perseverative thinking, ruminative tendencies, and depressive symptom severity. [26] Further studies have been conducted showing that mindfulness increases sustained attention with a correlated increase in DMN-FPCN anticorrelations. [27, 28] However, it has not yet been demonstrated these same principles an ADHD-specific cohort, i.e., with individuals that have been diagnosed with ADHD according to current clinical standards.

ADHD symptoms, including inattention, create barriers to patient progress in successfully acquiring and utilizing mindfulness strategies, however a number of studies suggest that self-empowerment and reward reinforcement are highly effective in modulating the attentional deficits of children with ADHD. [29, 30] It is hypothesized that real time neurofeedback, in particular the ability to visualize self-regulation of brain network activity patterns will help motivate ADHD patients to adopt and sustain mindfulness practices. Given that greater degrees of mind-wandering in ADHD is associated with more severe clinical features (e.g., inattention, executive function, emotional dysregulation, quality of life) [31, 32] a treatment targeting mind-wandering holds promise toward improving multiple, rather than single, symptoms and/or outcomes.

To develop personalized behavioral interventions that regulate attention in individuals with ADHD, an investigation focused on subjects who have been clinically diagnosed with ADHD though traditional channels (i.e., school-sanctioned counselor assessment or psychiatric assessment and diagnosis with the appropriate diagnostic manual) has been designed. For these individuals, modulation of the coupling between the DMN and FPCN, such that the FPCN is optimally engaged while the DMN is suppressed, will result in improvements in inattention symptoms and executive function in ADHD.

Embodiments leverage the power of real-time fMRI neurofeedback to inform highly individualized behavioral therapies and rebalance patterns of brain network activity that have become disordered. Embodiments relate to a technology-based Mind Balance Training (MBT) platform that supports individuals suffering from neuropsychiatric conditions, ultimately enabling them to recognize the onset of their own symptoms, initiate behavioral interventions that are targeted to their own precise neural architecture and activity patterns, and create brain network dynamics that are conducive to positive mental health. The training platform will help patients reach their full potential in the context of their lives, communities, and societies.

An example embodiment is a Mind Balance Training platform for the development of precision behavioral therapies. The method sequentially: (i) brings together individuals and medical practitioners to implement therapies in a technology intensive clinical setting; (ii) enables continued training in a more naturalistic (i.e., at-home) environment with less intensive technology-assistance, and ultimately, (iii) prepares individuals to incorporate precision behavioral interventions into everyday life without the use of technology. The Mind Balance Training embodiment comprises of two sub-systems, Mindscope and Mindwear. An aspect of the embodiments is a portfolio of behavioral interventions that incorporate both neural signatures of brain states, and the individual's experience of these states, in their formulation; these interventions may be included in the MBT platform.

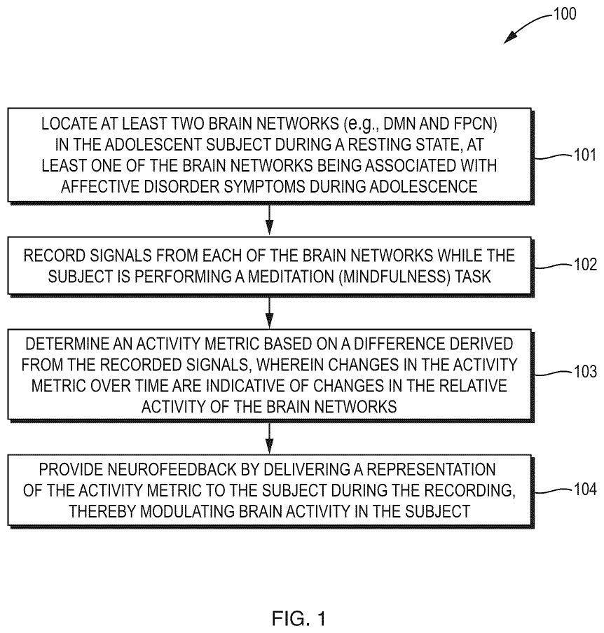

FIG. 1 shows an exemplary flow chart of a method 100 for modulating brain activity according to an embodiment of the invention. The method begins (101) by locating at least two brain networks (e.g., DMN and FPCN) in the adolescent subject during a resting state, at least one of the brain networks being associated with affective disorder symptoms during adolescence. Next, at 102, signals from each of the brain networks are recorded while the subject is performing a meditation (mindfulness) task. These signals may be blood-oxygen-level-dependent (BOLD) signals recorded using fMRI. At 103, an activity metric based on a difference derived from the recorded signals, wherein changes in the activity metric over time are indicative of changes in the relative activity of the brain networks. At 104, neurofeedback is provided by delivering a representation of the activity metric to the subject during the recording, thereby modulating brain activity in the subject. The modulating of brain activity may comprise downregulating or decreasing activity level in one of the brain networks. For example, BOLD signal activation. Likewise, the modulating may also comprise upregulating or increasing the activity level in one of the brain networks. For example, BOLD signal activation. The method 100 may further include determining a first resting state activity level before the performing of the meditation task, determine a second resting state activity level after the performing of the meditation task, and assess a change in functional connectivity of at least one of the brain networks based on a comparison between the first and second resting state activity levels.

An embodiment relates to Mindscope technology and involves developing individualized interventions in a magnetic resonance imaging (MRI) environment. Building on state-of-the-art approaches using fMRI to improve cognitive performance [11, 12] an example method: (i) maps the patient's neural activity to their personal neuroanatomical architecture to create a precise, individualized readout of activity patterns within and between brain networks; (ii) uses real-time fMRI (rt-fMRI) to perform event-contingent experience-sampling, in which a patient's connectivity patterns trigger on-screen questions and prompt them to input their subjective experience at that moment; (iii) applies machine-learning to connect the patient's suboptimal brain states and mental experiences to relevant interventions; and (iv) trains patients to use the recommended interventions. For example, in mindfulness training, patients will practice controlling and regulating their mental state while receiving visual feedback of their own brain network patterns in real time through the Real-time fMRI neurofeedback Enhanced Mindfulness (REMind) protocol. [13, 14, 15] With practice, patients will learn to recognize the association between their real-time neural signatures and their mental experience of those signatures and employ intervention approaches that restore healthy mental states.

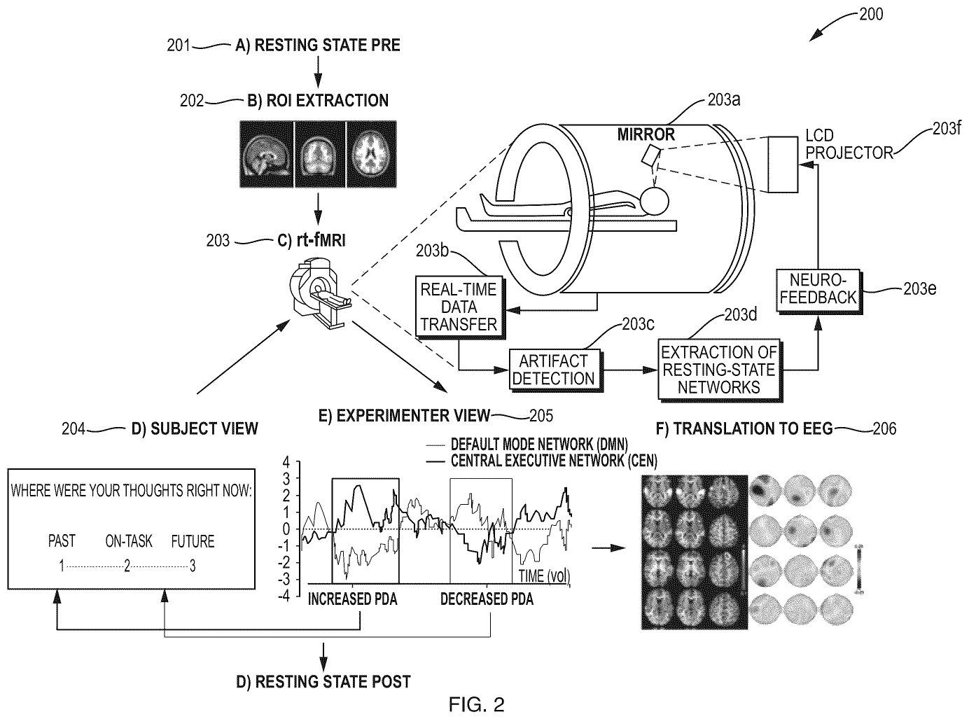

FIG. 2 shows an exemplary flow of an embodiment 200 of the invention relating to Mindscope. The method begins at identifying the resting state 201 of the patient's brain, which involves functionally locating brain networks by mapping the patient's neural activity to their personal neuroanatomical architecture to create a precise individualized readout of the activity patterns within and between the brain network. Example techniques or processes of functionally locating two brain networks are described in the article by Clemens et al, “Real-time fMRI neurofeedback reduces auditory hallucinations and modulates resting state connectivity of involved brain regions: Part 2: Default mode network-preliminary evidence,” Psychiatry Res. 284, 112770 (2020), the entire teachings of which are incorporated by reference. The method then moves to identifying the regions of interest (ROI) 202. This step is performed in a monitored setting using an rt-fMRI machine 203a. The rt-fMRI machine 203a comprises an MRI chamber with a mirror and LCD projector 203f, and facilitates real-time data transfer 203b, artifact detection 203c, extraction of resting-state networks 103d and neurofeedback 203e. The patient then begins meditation task. For example, in FIG. 2, the patient is shown on-screen questions 204 which prompt the patient to input their subjective experience at that moment. Signals (e.g., BOLD signals) from the brain are recorded while the subject performs these tasks. At this time, an experimenter observing the patient will be shown the default mode network and the cerebral executive network 205 of the patient. Then, the fMRI activity is translated into electroencephalography (EEG) signals 206. A Positive Diametric Activity (PDA) metric, based on the causal neural mechanism by which the central executive network (CEN) downregulates (or upregulates) the DMN, is used as a measurement of the relative activity level of the brain network. PDA is measured before and during mindfulness and is used to determine the brain activity to identify any changes in relative activity. This information can be used to provide neurofeedback to the patient. Additional details of the PDA metric can be found in Bauer 2020, incorporated by reference herein. For example, PDA can be calculated as (PDA=(CEN activation estimate)—(DMNactivation estimate)).

An embodiment relates to Mindwear technology and involves translating the interventions from Mindscope environments to a less controlled environment. After patients gain confidence in their ability to regulate their network activity through Mindscope training, which occurs in a highly supervised clinical setting and limits patient movements, they will progress to Mindwear, a light hardware that can be worn by the patient while moving and walking. Based on a showing that frequency specific components of electroencephalography (EEG) signals recorded on the scalp can serve as correlates of fMRI activity patterns [16], a software with EEG “fingerprints,” or signatures, of DMN-FPCN network dynamics may be further programmed with patient specific fMRI data to create individualized EEG signatures. As described in [16], in particular, the ability of the suggested framework to find an EEG Finger-Print (i.e. EEG features) that can predict the fMRI BOLD activity in the amygdala during real-time relaxation training guided by alpha/theta neurofeedback. In particular, it is shown that a ridge regression model, which is based on time/frequency representation of EEG data from a single electrode where each frequency band has its own delay, can predict the amygdala activity better than a linear combination of frequencies with a pre-defined delay and significantly better than traditional theta/alpha activity. Using Mindwear, patients will consolidate their ability to recognize suboptimal brain states through EEG signal training, and practice interventions in a more naturalistic setting. Employing recent advances in machine learning for bio-signals [17], Mindwear will provide continuous feedback of EEG fingerprints as a way for patients to evaluate, refine and improve their intervention strategies. Movement based interventions which are not feasible using Mindscope may also be studied using Mindwear, where the nature of the intervention (mindfulness, exercise, etc.) will depend on the specific symptom and its severity. Embodiments may be used at-home, on an as-needed basis determined by the clinician and patient. The EEG-based technology approaches may be used with other biometric devices. These methods may allow each afflicted person the ability to self-detect vulnerable brain states in the real world without the use of external aids, and initiate interventions on their own volition.

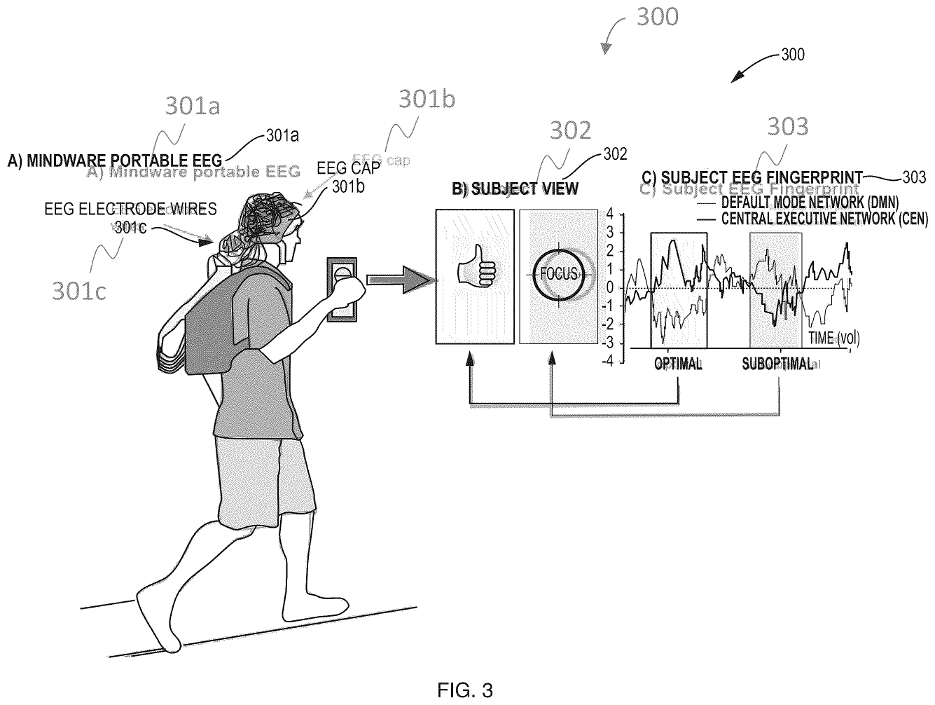

FIG. 3 shows an exemplary flow of an embodiment 300 of the invention relating to Mindwear. The system involves a portable Mindware EEG setup 301a, comprising of the EEG cap 301b atop the user's scalp, and the EEG electrode wires 301c. The system 300 is designed to be a portable, at home, version of the system 200 shown in FIG. 2 while using the individualized readout of their activity patterns created using the system 200. As such, the system 300 will use the personalized readout of the patient's activity patterns as a reference point. The system 300 measures and monitors the users brain networks via the portable EEG system 301a-c atop the user's scalp. The user will be shown subject view 302 for them to practice mindfulness interventions in a more naturalistic setting than that of Mindscope of FIG. 2. The EEG “fingerprints” are further programmed with patient specific fMRI data to create the individualized EEG signatures 303. This method and system provides neurofeedback to the user by allowing them to see their mind fullness in real time, and as such, modulate their brain activity.

An embodiment involves connecting brain states to subjective experience. An example method builds on the established practice of experience sampling (i.e., self-reports of behavior, emotion, or experience collected at random points in time) by introducing real time fMRI-initiated experience sampling. As opposed to collecting self-reports at random intervals, embodiments will initiate self-report questions at each point that a specific connectivity pattern of interest arises. This convergence of signaling activity and patient experience allows sampling of mental experiences that are highly salient to the individual and strengthens evidence for causal mind-brain relationships. High-speed computational analyses can be employed to discover predictive relationships between neural signatures and individuals' subsequent mental states. The self-generated experience as described by the patient can eventually serve as a reference point for patients indicating they should initiate an intervention and/or helping them recognize they are achieving a balanced mental state.

An embodiment involves utilizing deeply individualized interventions. Potentially, anti-correlation brain patterns may vary widely among individuals from diverse racial, ethnic, socio-economic and/or other environment-specific contexts. A recent study has gained national attention for highlighting major harmful gaps in the efficacy of widely utilized predictive models of suicide risk in hospitals, which were shown to work disproportionately well for Caucasian adolescents and children but performed poorly for African-American adolescents. [18] Furthermore, evidence has shown that children with low-income backgrounds [19] who perform well on cognitive tests do not exhibit the same neural correlates as those with higher income backgrounds. Advancements in this field of technology, indicates that environmental factors influence how individuals engage large scale brain networks to perform behavioral tasks. In designing individualized interventions, embodiments (e.g., the mind balance training platform) will treat all variations of baseline, resting state activity patterns, vulnerable brain states, and healthy brain states, as structured and meaningful information that informs the personalized interventions that will be delivered.

An advantage of embodiments relates to volitional control of brain activity (partnering with patients). The Mindscope methods & the REMind protocol leverage a novel method, which enables the individual to visualize their own brain activity in real time. [11, 13] The equipment setup includes a monitor within the scanner's interior chamber that displays a dynamic visual model reflecting patients' neural activity. Patients are trained to recognize their own sub-optimal and optimal brain states. When practicing an intervention, such as mindfulness, patients will see evidence of their own ability to influence their brain activity as their dynamics shift from suboptimal toward optimal. Patients tend to experience that volitional control of one's own brain network is empowering for many individuals practicing this method. For example, patients who have previously had difficulty sustaining mindfulness interventions, or practicing mindfulness, have become both motivated and skilled practitioners of this technique.

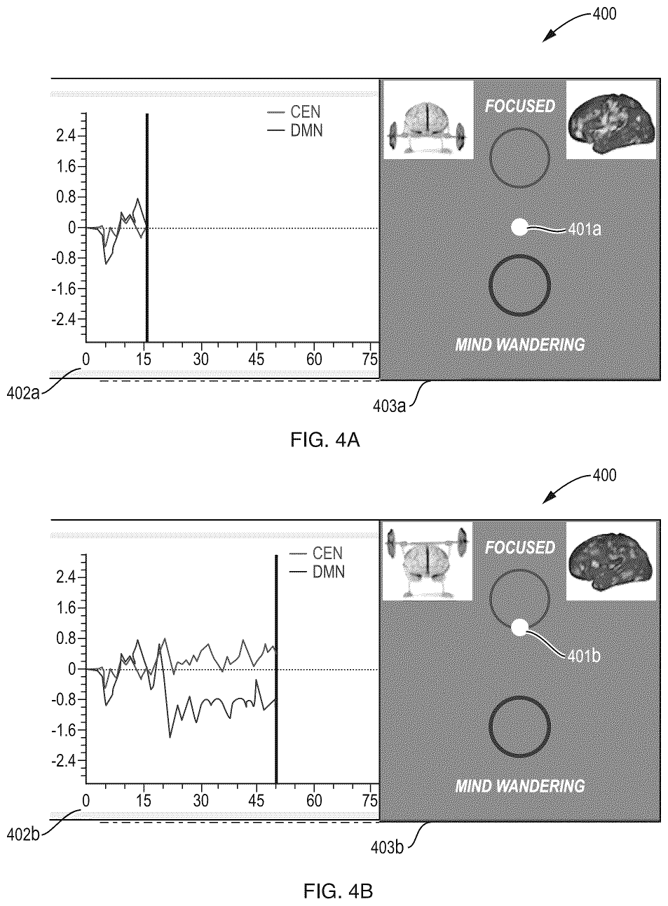

FIGS. 4A and 4B show a visualization of brain activity during the REMind protocol training process 400 according to an embodiment of the invention. The process provides neurofeedback to the individual by allowing them to visualize their own brain activity in real time as a “game.” The graphs 402a-b show the CEN and DMN measurement levels to a clinician chaperoning the testing, and the perspectives shown by 403a-b are what the patient sees. The patient monitors the white dot 401a-b on a screen while connected to neurofeedback systems described herein. While the patient's brain activity is not yet controlled by subject the center white dot 401a hovers idle in the center, as illustrated in FIG. 4A. However, as the subject begins to focus, the white dot 401b has moved towards the “Focused” target while the activity level of the brain networks are being monitored by the REMinds system, as illustrated in FIG. 4B. This gives the user a visual representation of their mindfulness, allowing them to identify their focused mental states via practicing mindfulness exercises which move the ball upwards, and learn to control their mindfulness.

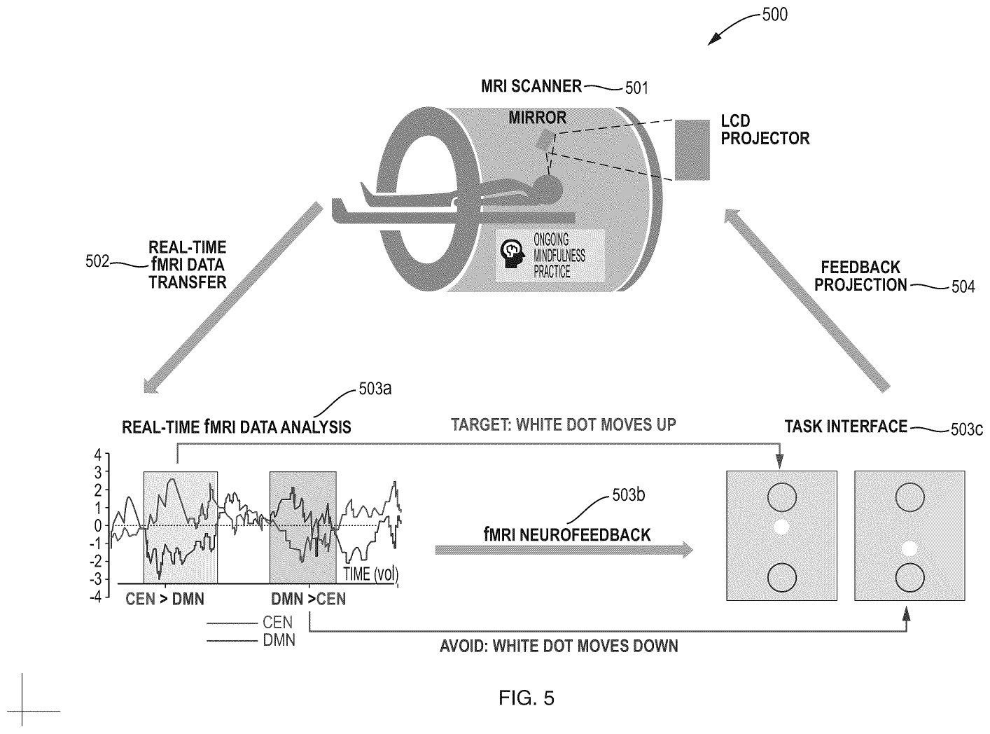

FIG. 5 shows an embodiment of a system 500 where the REMind protocol “game” of moving the white dot as in FIGS. 4A-4B can be used with the Mindscope methods and systems, where the patient is stationary in the rt-fMRI machine. In the system 500, a patient will be instructed to practice mindfulness to move the white dot on the screen up into the red circle. The movement of the white dot is dependent on real-time analysis of the fMRI data that computes the difference in personalized DMN and CEN activations. When DMN activation is lower than CEN activation, the white dot moves up; when DMN activation is higher than CEN activation, the white dot moves down. In the system 500, the brain networks are and recorded, and an activity metric determined, in the same manner as the system 200. In this feedback loop, the real-time fMRI data transfer 502 from the patient in the MRI scanner 501 gets transformed into the real-time fMRI data analysis 503a, the fMRI neurofeedback 503b transforms this into the usable task interface 503c, and the feedback projection 504 goes back to the user in the MRI scanner 501. This way, the patient receives a visual representation of neurofeedback via the REMind protocol, while being chaperoned in a clinical environment.

Another example advantage of embodiments relates to cost containment (health equity). Considerable focus is invested on developing a platform that can reliably prescribe individualized behavioral interventions over individualized pharmaceutical interventions. While each will initially require skilled professionals and specialized resources, accompanied by a training period (and potentially check-ups), the Mindwear systems and methods of the training program will be able to be practiced at home, which dramatically increases the ability of the broader population to access these therapies. Thus, unlike pharmaceuticals, the embodiments training platform interventions are highly individualized, and will not generate off-target side effects, and eventually are free of cost.

A further example advantage of embodiments relates to transdiagnostic scaling (partnering with patients, health equity). The mind balance training platform systems and methods have the potential to improve mental wellness among individuals afflicted with a variety of mental illnesses (e.g., anxiety, depression, psychosis), as well as children at-risk for developing mental illness. Extensive empirical and review work that implicates the DMN-FPCN network dynamics leveraged in Mindscope embodiments as a transdiagnostic marker of symptom improvement across a variety of domains. [20] It has been shown that real-time neurofeedback training in schizophrenic patients with severe auditory hallucinations reduced symptoms after just a single session [13] and are currently completing a pilot study to test the efficacy of the intervention in adolescent patients with anxiety/depression. [21] For those with ADHD, the training platform can be used to specifically improve cognition, such as sustained attention and executive function. Using this robust, modular, and adaptable platform, embodiments offer an opportunity for all types of individuals to visualize and volitionally control their brain network architecture as they perform behavioral interventions, and to accelerate intervention skills through technology assisted practice.

Embodiments may benefit numerous clinical populations (e.g., anxiety, ADHD, depression, psychosis) as well as youth who may be at-risk for developing mental illness as it allows individuals to volitionally control their brain network architecture and subsequently mitigate clinical symptoms/vulnerable brain states (e.g., anxiety, depression, stress, suicidal ideation, inattention, etc.) and increase cognitive performance.

For example, Mindscope embodiments can be used to track positive and negative symptoms (e.g., hallucinations) in schizophrenia, as well as depression symptoms (e.g., rumination) and help determining how to optimally modulate activities in associated brain networks in MRI scanner. Mindwear methods and systems can be used to translate the neuromodulation from MRI scanner to EEG.

For example, Mindscope embodiments can be used to track Attention deficit/hyperactivity disorder (ADHD) symptoms (e.g., inattention) and help determining how to optimally modulate activities in associated brain networks in MRI scanner. Mindwear embodiments can be used to translate the neuromodulation from MRI scanner to EEG.

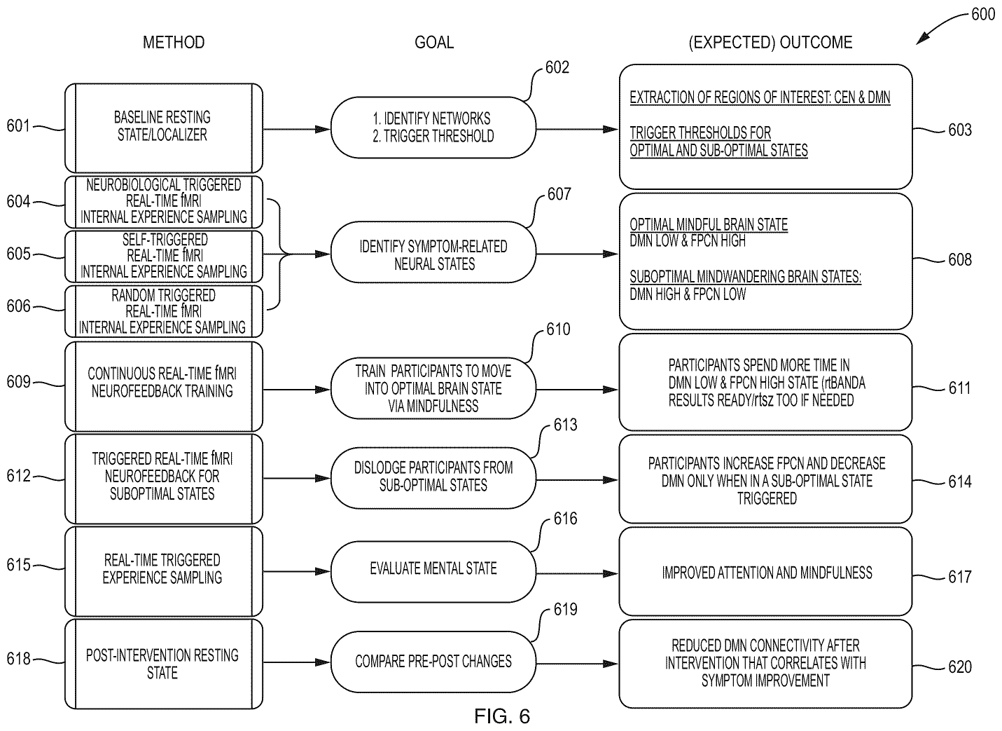

FIG. 6 is an example workflow 600 relating to the expectations and outcomes of various steps of the embodiment. The method starts by identifying the baseline resting state 601. The goal of 601 is to identify 602 the brain networks and the trigger threshold on the patient. The expected outcome 603 is extraction of regions of interest (CEN and DMN) as well as to trigger a threshold for optimal and sub-optimal brain states. The objective of neurobiological triggered real-time fMRI internal experience sampling 604, self-triggered real-time fMRI internal experience sampling 605, and random triggered real-time fMRI internal experience sampling 606 is to identify 607 symptom related neural states. The outcome 608 is to find optimal mindful brain states (DMN low and FPCN high) and suboptimal mind wandering brain states (DMN high and FPCN low). The goal of continuous real time fMRI neurofeedback training 609 is to train 610 participants to move into optimal brain states via mindfulness, and thus the participants spend more time in DMN low and FPCN high states 611. Triggering 612 the real time fMRI neurofeedback for optimal states dislodges 613 participants from sub-optimal states. The participants increase 614 FPCN and decrease DMN only when a sub-optimal state is triggered. The real time triggered experience sampling 615 compares 616 the evaluated mental state and leads to an improved 617 attention and mindfulness in the patient. Post-intervention resting state 618 compares 619 the changes from pre-intervention to post-intervention, and the reduced 320 DMN connectivity after intervention that correlates with symptom improvements.

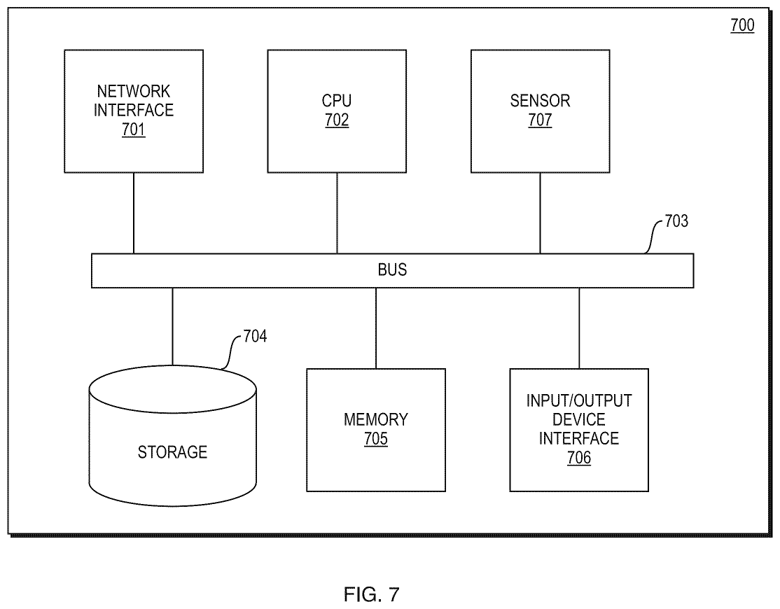

FIG. 7 is a simplified block diagram of a neurofeedback system 700 that may be used to modulate brain activity in an adolescent subject, for example, as in the method 100 of FIG. 1. The system 700 comprises a bus 703. The bus 703 serves as an interconnect between the various components of the system 700. Connected to the bus 703 is an input/output device interface 706 for connecting various input and output devices such as a keyboard, mouse, display, speakers, etc. to the system 700. A central processing unit (CPU) 702 is connected to the bus 703 and provides for the execution of computer instructions implementing embodiments. Memory 705 provides volatile storage for data used for carrying out computer code instructions implementing embodiments described herein. Storage 704 provides non-volatile storage for software instructions, such as an operating system (not shown) and embodiment configurations, etc. A sensor 707 provides the system with data relating to the patient or user. The sensor 707 may one sensor or many sensors, the sensor 707 may be a plurality of configurations of sensors designed to provide data relating to the patient. The system 700 also comprises a network interface 701 for connecting to any variety of networks known in the art, including wide area networks (WANs) and local area networks (LANs).

It should be understood that the example embodiments described herein may be implemented in many different ways. In some instances, the various methods and machines described herein may each be implemented by a physical, virtual, or hybrid general purpose computer, such as the computer system 700. The computer system 700 may be transformed into the machines that execute the methods described herein, for example, by loading software instructions into either memory 705 or non-volatile storage 704 for execution by the CPU 702. One of ordinary skill in the art should further understand that the system 700 and its various components may be configured to carry out any embodiments or combination of embodiments described herein. Further, the system 700 may implement the various embodiments described herein utilizing any combination of hardware, software, and firmware modules operatively coupled, internally, or externally, to the system 700. Further, the system 700 may be communicatively coupled to or be embedded within a manufacturing device so as to control the device to create a physical object as described herein.

### EXEMPLIFICATION

The mindfulness-based fMRI neurofeedback method and example results presented below have been described in the article by Jiahe Zhang et al., “Reducing default mode network connectivity with mindfulness based fMRI neurofeedback: a pilot study among adolescents with affective disorder history,” Molecular Psychiatry; https://doi.org/10.1038/s41380-023-02032-z, published online 30 Mar. 2023, the entire teachings of which are incorporated herein by reference.

Adolescents experience alarmingly high rates of major depressive disorder (MDD); however, gold-standard treatments are only effective for ˜50% of youth. Accordingly, there is a critical need to develop novel interventions, particularly ones that target neural mechanisms believed to potentiate depressive symptoms. Directly addressing this gap, a mindfulness-based fMRI neurofeedback (mbNF) is developed for adolescents that aims to reduce default mode network (DMN) hyperconnectivity, which has been implicated in the onset and maintenance of MDD. In this proof-of-concept study, adolescents (n=9) with a lifetime history of depression and/or anxiety were administered clinical interviews and self-report questionnaires, and each participant's DMN and central executive network (CEN) were personalized using a resting state fMRI localizer. After the localizer scan, adolescents completed a brief mindfulness training followed by a mbNF session in the scanner wherein they were instructed to volitionally reduce DMN relative to CEN activation by practicing mindfulness meditation. Several promising findings emerged. First, mbNF successfully engaged the target brain state during neurofeedback; participants spent more time in the target state with DMN activation lower than CEN activation. Second, in each of the nine adolescents, mbNF led to significantly reduced within-DMN connectivity, which correlated with post-mbNF increases in state mindfulness. Last, a reduction of within-DMN connectivity mediated the association between better mbNF performance and increased state mindfulness. These findings demonstrate that personalized mbNF can effectively and non-invasively modulate the intrinsic networks associated with the emergence and persistence of depressive symptoms during adolescence.

### Introduction

In the United States, major depressive disorder (MDD) results in ˜$200 billion of lost productivity and health care expenses annually [A1], and rates among adolescents are alarmingly high [A2]. Gold-standard treatments for depression are only effective for ˜50% of youth [A3], underscoring the critical need to develop novel treatments to improve clinical outcomes.

At the neural systems level, MDD is characterized by elevated resting state connectivity within the default mode network (DMN), which includes core midline hubs in the subgenual anterior cingulate cortex (sgACC), medial prefrontal cortex (MPFC), and posterior cingulate cortex (PCC) [A4, A5]. Although the sgACC is not typically considered a DMN node in ICA-based analyses (likely due to its low signal-to-noise ratio compared to other major DMN nodes at 3 Tesla), seed-based connectivity analyses have consistently placed the sgACC within canonical DMN topography (e.g., [A6, A7]). DMN hyperconnectivity, especially sgACC hyperconnectivity, is associated with symptom severity in depressed individuals [A8, A9] and characterizes children with elevated familial risk for depression [A10]. The DMN is thought to facilitate patterns of depressogenic, self-referential processing and a heightened focus on distressing emotional states [A4, A11-A13]. In MDD, it is theorized that dysregulation of the DMN by top-down control networks, such as the central executive network (CEN) [A14, A15], also contributes to heightened self-focus. Accordingly, hyperconnectivity within the DMN has been linked to rumination (i.e., the tendency to perseverate about one's symptoms), a common trait that contributes to depression onset, maintenance, and recurrence [A16-A18] as well as cognitive therapy non-response and relapse [A17, A19].

As DMN connectivity is a promising biomarker of MDD [A9, A20], new interventions targeting DMN have been explored. For example, transcranial magnetic stimulation (TMS) targeting the dorsolateral prefrontal cortex (i.e., a CEN node that is anticorrelated with DMN) normalizes DMN connectivity and improves depressive symptoms in adults [A21]. Interestingly, mindfulness meditation also can lead to decreased DMN activity [A22-A26] and connectivity [27], and importantly, improves depression treatment outcomes [A28-A31]. Although research has demonstrated that adolescents can apply mindfulness practices to reduce depression symptoms [A32, A33], certain depressive symptoms (e.g., inattention, lack of energy, apathy) may prevent adolescents from more successfully integrating and applying mindfulness strategies in daily life.

ADHD=attention-deficit/hyperactivity disorder, MFQ=Mood and Feelings Questionnaire, OCD=obsessive-compulsive disorder, RCADS=Revised Child Anxiety and Depression Scale.

To facilitate the acquisition and utilization of mindfulness strategies, a novel mindfulness-based fMRI neurofeedback (mbNF; [A34]) approach was developed, which is a non-invasive technique that allows people to track and modulate brain function. To date, neurofeedback studies in depression have frequently involved mood-related tasks, such as negative emotion induction or valenced autobiographical memory recall (see review in [A35]). By contrast, the mbNF embodiment aims to reduce DMN connectivity given associations with mindfulness and MDD. In this 15-min neurofeedback paradigm, people observe a schematic visual representation of their brain activity and practice mindfulness to volitionally reduce DMN relative to CEN activation. It has been demonstrated that mbNF reduced DMN connectivity in adults with schizophrenia and led to symptom reduction post-intervention [A36]. As this mbNF method is non-invasive and optimizes the implementation of mindfulness to reduce DMN connectivity, it has enormous potential to facilitate skill acquisition outside of the scanner.

Building on fMRI neurofeedback research [A36, A37], the feasibility of mbNF in adolescents with a history of affective disorders was tested in this non-randomized, single-arm, proof-of-concept study. First, it was tested whether adolescents would spend more time in the target state, characterized by lower DMN than CEN activation. Second, it was tested whether mbNF leads to reduced DMN connectivity and associated increases in state mindfulness. Third, it was tested whether reduced DMN connectivity accounted for the association between successful neurofeedback and increased state mindfulness.

### Methods and Materials

### Participants and Procedure

The participants consisted of adolescents (n=9; 18.8±0.7 years; 17-19 years; 66.7% females) who previously completed scans for the Boston Adolescent Neuroimaging of Depression and Anxiety Human Connectome project (BANDA; [A38, A39]) were re-contacted, screened, and enrolled in this proof-of-concept study. All participants reported a lifetime history of MDD and/or anxiety disorders. Three participants exhibited current diagnoses; two reported clinical levels of MDD and anxiety disorders and one reported anxiety disorder only. Neurofeedback performance or post-neurofeedback changes did not differ based on the current vs. lifetime presence of diagnoses. A summary of the participants' sociodemographic and clinical characteristics can be found in Table 1 of Jiahe Zhang et al., Molecular Psychiatry, 2023.

For Session 1, which was a follow-up to the BANDA protocol, study procedures were approved through the Mass General Brigham TRB. At the baseline visit, participants were administered a clinical interview and self-report assessments of depressive and anxiety symptoms. Then, each participant completed a localizer MRI session at the Athinoula A. Martinos Center for Biomedical Imaging. At the end of Session 1, participants were provided with information for Session 2 and interested participants were enrolled. Session 2 procedures were approved by the Northeastern University Institutional Review Board, and typically occurred within 2-3 weeks of Session 1. Participants underwent mindfulness meditation training (15 min), completed a neurofeedback MRI session at the North-eastern University Biomedical Imaging Center (1 h), and completed pre- and post-scan state mindfulness assessments (10 min). With transitions and other tasks/assessments not related to the current analyses, Session 2 typically lasted 2.5 h. Informed consent was obtained from all subjects for both sessions of the study.

### Session 1

Participants were administered the Kiddie Schedule for Affective Disorders and Schizophrenia Present and Lifetime Version (KSADS; [A40]) to provide an assessment of psychiatric disorders as of their most recent study visit, which had occurred 2-3 years prior. Participants also completed the 33-item Mood and Feelings Questionnaire (MFQ; [41]) to assess depression symptom severity. Total scores range between 0-66, with higher scores indicating more severe depression symptoms. Participants also completed the Revised Child Anxiety and Depression Scale (RCADS; [A42]). The primary subscales of interest characterize general anxiety and social anxiety symptoms.

MRI data were acquired on a Siemens Prisma scanner with a 64-channel, phased-array head coil (Siemens Healthcare, Erlangen, Germany), including: a T1-weighted MPRAGE structural scan [0.8 mm isotropic voxel size, 208 slices, field-of-view (FOV)=256×240×167 mm, repetition time (TR)=2400 ms, echo time (TE)=2.18 ms, flip angle (FA)=8° ] and two resting state fMRI scans (rs-fMRI: 5 min 46 s each, eyes open with fixation, multiband acceleration factor=8, 2 mm isotropic voxels, 72 slices, FOV=208×208×144 mm, TR=800 ms, TE=37 ms, FA=52°) to identify participant-specific DMN and CEN maps.

Preprocessing of rs-fMRI data was performed in FSL 6.0 [A43] including: motion correction, brain extraction, co-registration, smoothing and bandpass filtering (see more details in [A36]). An independent components analysis (ICA) was performed on concatenated preprocessed functional scans using Melodic ICA v3.14 [A1] with dimensionality estimation using the Laplace approximation to the Bayesian evidence of the model. Each of the ˜30 spatiotemporal components were statistically compared to atlas spatial maps of the DMN and CEN derived from rs-fMRI of ˜1000 participants [A2] using FSL's “fslcc” tool and the ICA components that yielded the highest spatial correlation for each participant were selected. ICA components were thresholded to select the upper 10% of voxel loadings and binarized to obtain participant-specific DMN and CEN masks for neurofeedback in Session 2. Visual inspection was performed, and all selected components were determined to be satisfactory in covering canonical DMN (2436.11±328.78 voxels) and CEN (2315.00±105.09 voxels) brain regions [A44]. There was no significant difference between the number of voxels in DMN vs. CEN masks (p>0.05, two-tailed paired-sample t-test).

### Session 2

In Session 2, participants completed: pre-mbNF state mindfulness assessment, mindfulness training, structural scan, pre-mbNF rs-fMRI, mbNF, post-mbNF rs-fMRI, post-mbNF state mindfulness assessment.

Participants completed the State Mindfulness Scale (SMS; [A45]) by indicating on a 5-point scale their perceived level of awareness and attention to their present experience during the last 15 min. The SMS was scored both as a sum of all 21 items (ranges 0-105) as well as two subscales assessing 15 items on mindfulness of the mind (i.e., thoughts and emotions; ranges 0-75) and 6 items on mindfulness of the body (i.e., movement and physical sensations; ranges 0-30).

Participants were trained on a mindfulness technique called “mental noting”. Mental noting is a major component of Vipassana [A46] and consists of the factors “concentration” and “observing sensory experience.” The experimenter explained that mental noting entails being aware of the sensory experience without engaging in or dwelling on the details of the content; in other words, one would “note” the sensory modality (e.g., “hearing,” “seeing,” “feeling”) at the forefront of their awareness and then let it go after it has been noted. The experimenter also introduced the concept of an “anchor”, or a sensory experience to which one could easily switch their attention, such as breathing. Participants were encouraged to use their personal “anchors” when they noted consecutive “thinking” (i.e., rumination). The experimenter demonstrated noting out loud by verbalizing the predominant sensory modality approximately once per second. Participants were then asked to practice mental noting out loud to demonstrate the ability to describe sensory awareness without engaging in the content and stop consecutive “thinking”.

To assess the effectiveness of mindfulness training, participants listened to audio recordings of brief stories before and after training. This included five stories describing everyday, mundane characters and events recorded in neutral tone. Each story lasted about 30 s and included 20 unique details. For example, the sentence “Grandpa owns a garden” includes 3 unique details (i.e., “grandpa,” “owns,” “garden”). Immediately before training (i.e., baseline), they listened to one story and were asked to freely recall as many details as possible. After training, participants were asked to practice mental noting while stories were played (i.e., introducing salient auditory stimulus). The number of stories ranged between 2-4 and was stopped either after the participant was comfortable at the practice or after all remaining 4 stories were played. Compared to the baseline test when participants fully attended to story playback, usage of noting strategy during playback led to a significantly reduced number of details recalled [t(8)=−16.20, p<0.001, two-tailed paired-sample t-test], indicating that participants were successfully engaging the noting strategy and not retaining the details of the story.

A multivariate and univariate real-time functional imaging (MURFI), an open-source software package to support rs-fMRI neurofeedback was used. Detailed user manual can be found in [A34] and online (https://github.com/cccbauer/MURFI-user-manual). The neurofeedback system is a network of TCP/IP-connected computers including the Siemens MRI scanner, a high-performance Linux laptop running MURFI, and a Windows 10 laptop running stimulus presentation using PsychoPy [A47]. During neurofeedback scans, echo-planar imaging (EPI) volumes are continuously reconstructed and transferred from the scanner to the MURFI computer via the TCP/IP connection.

Neurofeedback is a type of biofeedback that provides feedback signal(s) (e.g., visual) based on activation in target brain regions to teach participants to self-modulate brain activation. During neurofeedback, a first displayed a centered crosshair for a 30-s baseline was played and instructed participants to rest. Then, a continuous 2-min block started with three non-overlapping display items aligned on a vertical axis of a gray screen: a central white dot (x=0, y=0, radius=12 pixels), a red circle above (x=0, y=1, radius=56 pixels) and a blue circle below (x=0, y=−1, radius=56 pixels) (FIG. 5). The distance between the centers of the red and blue circles was 472 pixels. The visual feedback was displayed by movements of the white dot. To achieve the visual feedback, MURFI used all data acquired to fit an incremental general linear model (GLM; [48]), where linear trend nuisance signals were discounted and scaled activation estimates were computed for each voxel within each network-of-interest (NOI) (i.e., binarized DMN/CEN mask). It then combined activation across all NOI voxels using the weighted average method. The resulting activation estimate, in units of standard deviations from baseline, per NOI, was immediately sent to the stimulus computer where PsychoPy computed a positive diametric positivity (PDA) metric following the formula described in [A27, A36]: PDA=NOI(α)activation estimate—NOI(β) activation estimate. This PDA metric is based on the hypothesis that there is a causal neural mechanism by which the CEN downregulates the DMN [A49]. For each TR, the newly calculated PDA value was added to the y-value from the previous TR and the updated y-value was rendered as the new position of the white dot. Visually, the white dot would move upwards with positive PDA values and downwards with negative PDA values. The time delay between collection of a complete EPI volume and its associated position update was 30.5 s, and the position updated every TR. Once a participant had accumulated 5 TRs where the white dot was within or beyond a circle, the radius of the corresponding circle shrank by 10% (to titrate difficulty based on performance), and the white dot was repositioned to y=0. The circle would only shrink up to 5 times during one single neurofeedback run.

During mbNF, (referring to FIG. 5) participants were instructed to practice mindfulness to move the white dot on the screen up into the red circle. The movement of the white dot was dependent on a real-time analysis of the fMRI data that computed the difference in personalized DMN and CEN activations. When DMN activation is lower than CEN activation, the white dot moves up; when DMN activation is higher than CEN activation, the white dot moves down.

There were five back-to-back neurofeedback runs (2.5 min/run). Participants were instructed to move the white dot into the red circle by performing mental noting. Participants were instructed that upward movement of the dot was associated with effective mental noting performance and downward movement with ineffective mental noting, such as self-related processing and mind-wandering. This instruction was provided so that participants can anchor their subjective experience of engaging in mental noting to the observed movements of the white dot. Participants were aware that sustained positions within the red or blue circle (or beyond) would shrink the corresponding circle and return the white dot to the center. After each feedback run, it was confirmed whether participants had used mental noting during neurofeedback.

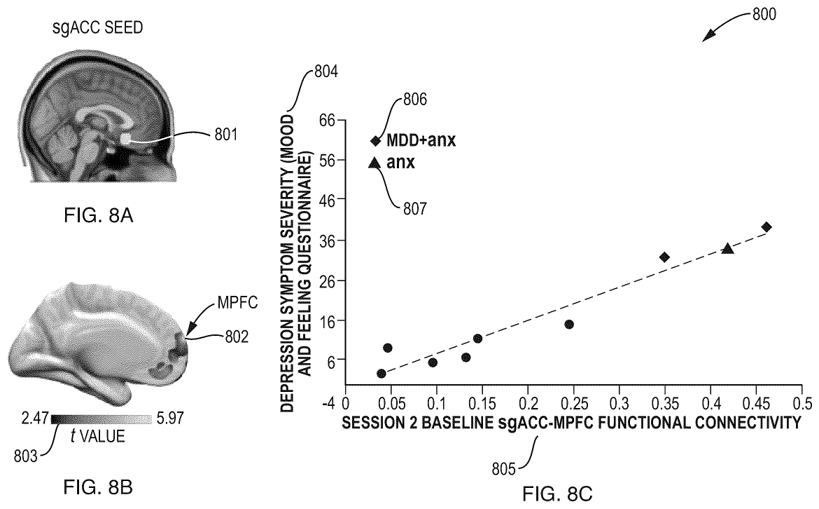

FIGS. 8A-8C illustrate the results of an experiment 800 showing that higher DMN functional connectivity was associated with more severe depression symptoms at baseline. Referring to FIG. 8A, an 8 mm spherical seed 801 in the sgACC was used [57]. Referring to FIG. 8B, functional connectivity between the sgACC seed 801 and the MPFC positively correlated with symptom severity. Higher MFQ score indicates higher severity. Arrow 802 indicates the peak of the MPFC cluster that survived p=<0.001 (uncorrected). FIG. 8B is displayed at p<0.05 (uncorrected) and the color bar range 803 reflects minimum and maximum t values in the connectivity map. FIG. 8C is a scatterplot which illustrates the correlation between baseline MFQ (804) and baseline sgACC-MPFC functional connectivity (805) (computed using an averaged time course across all voxels in the significant MPFC cluster). All participants had a lifetime history of MDD and/or anxiety. Patients with current diagnoses are labeled with a diamond (806) for having comorbid anxiety and depression (“MDD+anx”) and a triangle (807) for having anxiety only (“anx”). Participants with previous diagnoses are only shown as circles.

### Session 2 MRI Acquisition, Preprocessing and Data Analytic Overview

A structural scan was acquired using a T1-weighted MPRAGE pulse sequence (1 mm isotropic voxel size, 176 slices, FOV=256×256×176 mm, TR=2530 ms, TE=46 ms, FA=7°). For functional images, including during mbNF, the BOLD signal was measured using a T2*weighted gradient-echo, EPI pulse sequence (2 mm isotropic voxels, 68 slices, 10% gap, FOV=256×256×149.4 mm, TR=1200 ms, TE=30 ms, FA=72°). Each neurofeedback run lasted 2 min and 30 s. Immediately before and after mbNF, two rs-fMRI scans (5 min each, eyes open without fixation) were acquired.

Preprocessing was performed using fMRIPrep 21.0.0 [50], which is based on Nipype 1.6.1 [51]. In short, preprocessing included realignment, co-registration, normalization, susceptibility distortion correction, segmentation of gray matter (GM), white matter (WM), cerebrospinal fluid (CSF), skull stripping, and confounds extraction. Visual quality control was performed on each preprocessed run.

Preprocessed data and confound time series were imported into the CONN Toolbox v20.b [A52] where outlier identification was performed with the Artifact Detection Tools (ART, www.nitrc.org/projects/artifact_detect). Volumes with global signal z>5 or framewise displacement>0.9 mm compared to the previous frame were flagged as outliers. Relatively lenient thresholds were used to retain data given the small sample size. Comparable results were noted with 8 participants at more stringent thresholds (global signal z>3 and framewise displacement>0.5 mm; see FIG. S2). In addition, in-scanner mean motion was defined as the mean framewise displacement [A43] and calculated separately for pre- and post-mbNF runs. Rs-fMRI runs were spatially smoothed with a 6 mm Gaussian kernel. A principal component analysis identified noise components from WM and CSF following CONN's[53] a CompCorr method [A54]. During denoising, the test regressed out the top 5 WM noise components, top 5 CSF noise components, 12 realignment parameters (3 translation, 3 rotation, and their first derivatives), linear drift and its first derivative, motion outliers, and applied a bandpass filter of 0.008-0.09 HZ. In line with previous research [A52, A55, A56], the DMN reliability between Sessions 1 and 2 was assessed. Within-DMN connectivity (average connectivity between all a priori DMN seeds defined in the CONN toolbox, including MPFC, PCC, and bilateral inferior parietal lobules) was highly stable across the two sessions (ICC=0.871, p=0.005).

Using the CONN toolbox [A52], functional connectivity analyses were performed seeding the sgACC (8 mm-radius sphere around MNI −2, 22, −16) [A57]. Functional connectivity was calculated as Fisher-transformed Pearson's correlation coefficient. It was verified that the sgACC seed showed connectivity to canonical DMN nodes in each participant [t(8)=7.09, p<0.001, two-tailed t-test]. For baseline brain-behavior correlation analysis, the whole brain was searched for regions where connectivity with the seed correlated with MFQ scores at p<0.001 (uncorrected). For functional connectivity change, SPM small volume correction was used to search midline DMN regions (MPFC and PCC nodes as defined in CONN toolbox DMN network) for voxels whose connectivity with the seed region changed significantly after mbNF and reported clusters that survived a FDR-corrected threshold of q=0.05. Both analyses controlled for framewise displacement [58], and framewise displacement change did not correlate with SMS change (r=−0.35, p=0.35). MPFC-seeded analyses also were computed (8 mm-radius sphere around MNI −1, 53, −3) [A59]. No outlier was identified for measures used in correlation and mediation analyses.

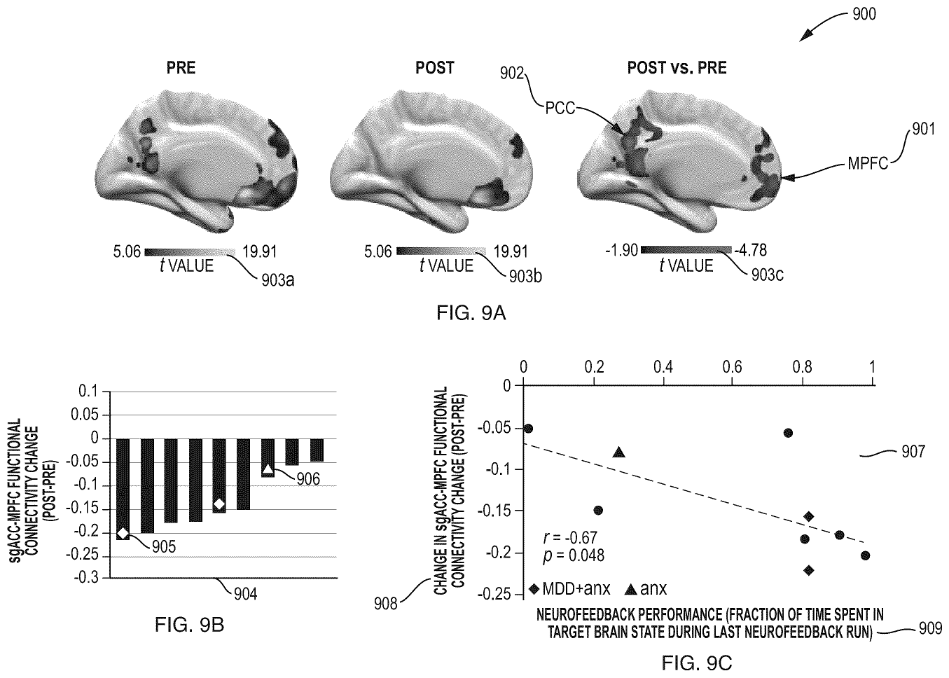

FIGS. 9A-9C illustrate the results 900 of an experiment where one session of mbNF reduced DMN functional connectivity. FIG. 9A shows that a t-test revealed that after mbNF, there was reduced connectivity between sgACC seed and midline DMN regions. Arrows indicate peaks in MPFC 901 and PCC 902 that survived q_{FDR}<0.05 (uncorrected). Color bar ranges 903a-c reflect the minimum and maximum t values in the maps. FIG. B shows a bar graph 904 which illustrates that reduced sgACC-MPFC connectivity was found in all participants. Each bar represents the change in functional connectivity strength in a participant. All participants had a lifetime history of MDD and/or anxiety. Patients with current diagnoses are labeled with a diamond 905 for having comorbid anxiety and depression (“MDD+anx”) and a triangle 906 for having anxiety only (“anx”). FIG. 9C shows a graph 907 illustrating a reduced sgACC-MPFC connectivity (908, Y-Axis) (computed using an averaged time course across all voxels in the significant MPFC cluster) was only associated with better neurofeedback performance (909, X-Axi). Participants with previous diagnoses are only shown as circles in the scatterplot.

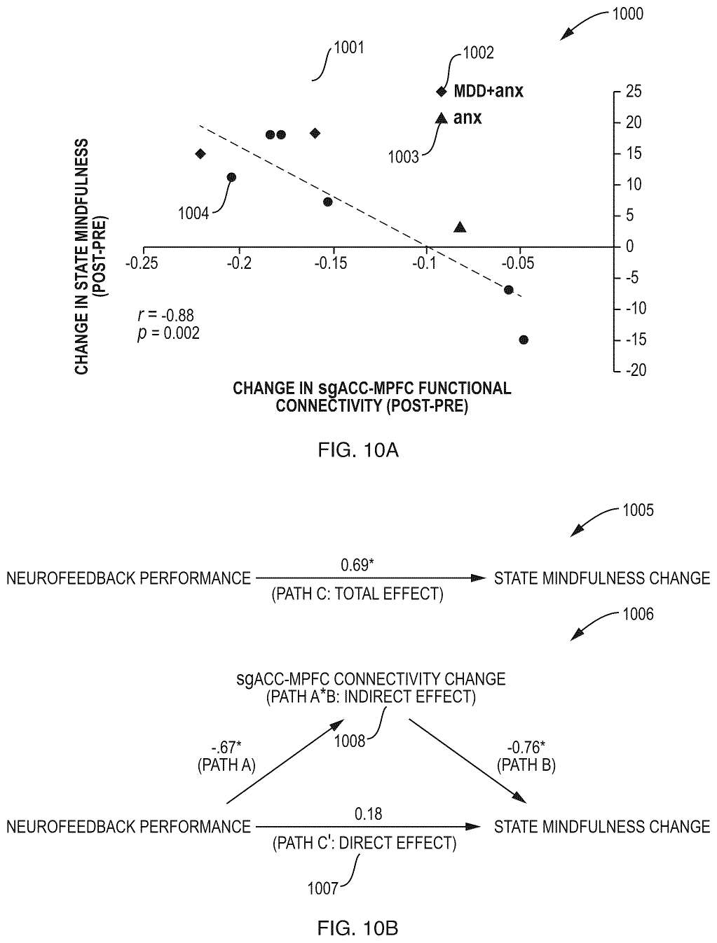

FIGS. 10A-10B illustrate the results 1000 of an experiment where one session of mbNF induced state mindfulness change. FIG. 10A shows a scatter plot 1001 which illustrates that a higher increase in state mindfulness after mbNF was associated with more decrease in sgACC-MPFC functional connectivity (computed using an averaged time course across all voxels in the significant MPFC cluster from FIG. 9A). All participants had a lifetime history of MDD and/or anxiety. Patients with current diagnoses are labeled with a diamond 1002 for having comorbid anxiety and depression (“MDD+anx”) and a triangle 1003 for having anxiety only (“anx”). Participants with previous diagnose are only shown as circles 1004. FIG. 10B shows a depiction which illustrates that reduction in sgACC-MPFC connectivity fully mediated the association between better neurofeedback performance and increase in state mindfulness. Arrows indicate paths, and path values indicate standardized beta weights. The upper panel 1005 shows the total effect (unmediated path c, total effect) from neurofeedback performance to state mindfulness change. In the lower panel 1006, the effect of neurofeedback performance on state mindfulness change is fully mediated by the change in sgACC-MPFC functional connectivity. The direct effect of neurofeedback performance to state mindfulness change is indicated by path c′ 1007 and the indirect effect is indicated by the ab path 1008 (i.e., path a*path b). p=0.05.

### Results

### Baseline DMN-Depressive Symptom Severity Association

Current depression symptom severity (MFQ) positively correlated (p<0.001, uncorrected) with baseline functional connectivity (i.e., pre-mbNF) between the sgACC seed and several regions, including the MPFC (21 voxels; peak at MNI −6, 66, 18), the right lateral temporal cortex (32 voxels; peak at MNI 58, −26, −10), and the middle frontal gyrus (28 voxels; peak at MNI 46, 46, 8). More severe depression symptoms were associated with greater connectivity between the sgACC seed (FIG. 8A) and several regions, including the MPFC (FIG. 8B), the right lateral temporal cortex (32 vocels; peak at MNI 58, −26, −10) and the middle frontal gyrus (28 vpce; s; peak at MNI 46, 46, 8). More severe depression symptons were associated with greater connectivity between the sgACC seed and the MPFC (FIG. 8C).

### Neurofeedback Performance

Averaging across all 5 neurofeedback runs, participants spent more time in the target brain state (DMN<CEN activation) than expected by chance (p=0.038, one-tailed t-test against 50% chance). Additionally, participants exhibited marginally lower, but non-significant, DMN activation than CEN activation (p=0.071, one-tailed paired-sample t-test).

### Functional Connectivity Change Following mbNF

To test changes in DMN functional connectivity following mbNF, sgACC seed functional connectivity was compared pre- vs. post-mbNF. At the group level, the sgACC seed showed significantly reduced functional connectivity (qFDR<0.05) to both MPFC (1003 voxels; MNI −8, 60, −6) and PCC (1185 voxels; MNI −4, −62, 28) after mbNF (FIG. 9A). Furthermore, when visualizing individual-level data, it was found that all nine participants showed sgACC-mPFC connectivity reduction (FIG. 9B). Additionally, a negative correlation between sgACC-MPFC connectivity change and neurofeedback performance was found. Participants who spent more time in the target brain state on the last neurofeedback run showed a greater reduction in sgACC-MPFC functional connectivity (r=−0.67, p=0.048; (FIG. 9C)). However, time spent in target state during the first 4 neurofeedback runs as well as average time spent in target state across all neurofeedback runs did not correlate with sgACC-MPFC functional connectivity change.

### Changes in State Mindfulness Pre- to Post-mbNF

Compared to pre-mbNF, participants reported significantly increased total state mindfulness after mbNF [t(8)=1.90, p=0.047], which was similarly observed in the mind [t(8)=1.56, p=0.079] and body subscales [t(8)=2.26, p=0.027] two-tailed paired-sample t-tests). As hypothesized, change in state mind-fulness was positively correlated with neurofeedback performance. Specifically, participants who spent more time in the target brain state on the final neurofeedback run showed greater increases in SMS total (r=0.69, p=0.039) as well as both the mind [r=0.71, p=0.031] and body subscales [r=0.59, p=0.093]. Further, a negative correlation between change in functional connectivity and change in state mindfulness was found. Relative to pre-mbNF, more reduction in sgACC-MPFC functional connectivity was associated with greater increases in state mind-fulness (SMS Total) following mbNF (r=−0.88, p=0.002; FIG. 10A); this association was consistent across the subscales (SMS Mind: r=−0.87, p=0.002; SMS Body: r=−0.82, p=0.007).

### DMN Functional Connectivity as a Mediator

Using cross-sectional mediation analysis [A60, A61], functional connectivity change partially mediated the association between neurofeedback performance and state mindfulness change. The state mindfulness change on neurofeedback performance was first regressed [b=23.22, β=0.69, t=2.53, p=0.039] (FIG. 10B; total effect, path c) and sgACC-MPFC connectivity change on neuro-feedback performance [b=−0.12, β=−0.67, t=−2.39, p=0.048](FIG. 10B; path a). Controlling for neurofeedback performance, the mediator (sgACC-MPFC connectivity change) significantly predicted state mindfulness change [b=−141.75, β=−0.76, t=−3.07, p=0.022] (FIG. 10B; path b). Further, controlling for the mediator (sgACC-MPFC connectivity change), neurofeedback performance was no longer a significant predictor of state mindfulness change [b=6.07, β=0.18, t=0.73, p=0.493] (FIG. 10B; path c). The Sobel test indicated that mediation by the indirect effect (path ab) approached significance (t=1.88, p=0.060).

### Discussion

Depression is one of the most common mental disorders among adolescents, resulting in severe impairments. Accordingly, there is an urgent need to develop novel treatments to address escalating rates of depression among adolescents. In this proof-of-concept study, several key findings emerged, which support the feasibility of using fMRI neurofeedback to target adolescent depression symptoms. First, participants successfully reduced DMN activation relative to CEN activation during mbNF. Second, one session of successful mbNF led to reduced DMN connectivity and increased state mindfulness. Last, a reduction in sgACC-MPFC connectivity mediated the association between enhanced neurofeedback performance and increased state mindfulness.

Neurofeedback is in its nascency phase, and accordingly, there are different ways to characterize success. In this study, during neurofeedback, although there were no differences in mean activation levels of DMN and CEN, participants spent more time in the target state of less DMN relative to CEN activation, indicating sustained effort in the expected direction. Additionally, continuous effort to regulate the brain during neurofeedback may lead to additional changes after the neurofeedback task. It is promising that mbNF significantly reduced resting state DMN connectivity and increased state mindfulness. Mindfulness practice, coupled with neurofeedback prompting participants to downregulate the DMN and upregulate the CEN, may help strengthen CEN's inhibitory control of the DMN [A49], which may improve the ability to deploy attentional resources to reduce repetitive self-referential thinking [A62].

The DMN consists of an ensemble of regions whose hyper-activation and hyperconnectivity result in altered self-referential processing and for some, may lead to rumination commonly occurring in MDD [A9, A20]. There are a number of neuroanatomical landmarks implicated in the DMN (e.g., MPFC, PCC, angular gyrus), and this seed-based analysis approach privileged the sgACC as it shows specific alterations in MDD, including reduced glial cell count [A67], abnormal blood flow and metabolism [A68, A69], abnormal thickness [A70, A71], reduced volume [A67, A68, A72], and structural connectivity [A73-A75]. Similar to previous studies [A8-A10], it was found that at baseline (i.e., prior to mbNF), elevated sgACC connectivity to other DMN nodes was associated with more severe depression symptoms. In the current study, the a priori sgACC seed also showed significant connectivity with individualized DMNs. However, it is worth noting that post-mbNF change in sgACC-MPFC connectivity, not MPFC-PCC connectivity, was specifically correlated with increased state mindfulness, suggesting that sgACC may be a special hub in depression treatment. This is consistent with theories suggesting that in MDD, hyperconnectivity between the sgACC and other midline limbic nodes of the DMN leads to ruminative response style [A9] and inefficient energy regulation [A76]. Accordingly, in other neuromodulation studies, MDD symptom improvement has been observed following deep brain stimulation specifically targeting the sgACC [A77, A78] as well as following TMS targeting the DLPFC subregion most anticorrelated with the sgACC [A79-A81], with normalized sgACC connectivity post-treatment [A21].

This study also provided evidence that after one mbNF session, reduced DMN connectivity mediated the association between neurofeedback performance and increased state mindfulness. This suggests that a change in DMN connectivity may be necessary to facilitate the behavioral change (i.e., state mindfulness) post-mbNF and is consistent with previous literature showing association between higher trait mindfulness and lower DMN connectivity [A82, A83]. In other words, dampening of DMN activity during mbNF and reduced DMN connectivity post-mbNF may provide favorable conditions for mindfulness acquisition. Similarly, a recent real-time neurofeedback study teaching healthy adolescents to regulate PCC activity using mindfulness meditation demonstrated increased state mindfulness immediately after the training as well as after 1 week [A84]. In both studies, participants down-regulated DMN activity with help from real-time neurofeedback and subsequently showed improvement in mindfulness, which may be a pathway to reduce negative repetitive thinking and depression symptoms.

One major innovation in this study is that instead of targeting a single brain region (e.g., the PCC), network-based modulation was implemented (i.e., DMN, CEN). As cognitive neuroscience has evolved from localized models to attributing function to distributed systems and their interactions, neurofeedback research has expanded from feedback targeting activation of a single region to include feedback based on multiple, related regions [A85]. Neuromodulation is a powerful tool because it can target distributed networks non-invasively, and in clinical settings, targeting distributed regions may lead to more effective outcomes than targeting a single region because many psychiatric disorders with heterogeneous phenotypes (including depression) show networked neuropathology [A86]. By modulating circuits, there may be a better chance to normalize altered brain connections and their associated functions. This is also one reason that circuit-based real-time fMRI neurofeedback may outperform focally targeted methods such as TMS and ultrasound.

Importantly, results showed that all 9 individuals who participated in the mbNF protocol demonstrated reduced DMN connectivity. Notably, this sample included 3 participants with current MDD and/or anxiety diagnoses, whose changes in DMN connectivity and SMS did not differ from the other participants, suggesting the current mbNF protocol may be fruitful as a transdiagnostic intervention. Future studies with larger sample sizes could examine patients with more severe symptoms as well as at-risk participants to explore differential effects on a range of psychiatric disorders. The ubiquitous DMN connectivity reduction may also be attributed to the personalized design of the protocol where the neurofeedback targets (i.e., DMN, CEN) were individually and functionally localized for each participant, which is in line with the broader mission in psychiatry toward utilizing biomarkers to guide precision medicine [A87]. By comparison, randomized controlled clinical trials for TMS—where a treatment target is typically not functionally localized-demonstrates a response rate between 15-37% and a remission rate between 14-30% [A88]. This proof-of-concept study is not a clinical trial, nor was longer-term clinical outcomes tested. However, personalizing treatment, which is common in deep brain stimulation [A89], may foster improved clinical outcomes for MDD. The added benefit of personalized neurofeedback targets can be formally tested, with a larger sample size, by comparing outcomes after implementing individualized vs. group-based neurofeedback targets.

There are several limitations in the current study. First, due to practical constraints, the current sample only included 9 participants. A larger sample is necessary for replicating and validating brain-behavior relationships. Second, the current pilot study is considered an early-phase exploratory trial to test the feasibility of the mbNF approach and thus a single-group design was justified [A90]. Due to the lack of a control condition, the test was unable to determine the unique effects of mindfulness and neurofeedback on the neural and behavioral changes observed. Similarly, the test was unable to assess any intervention bias such as placebo effect or demand characteristics. Future studies need to include a control condition where mindfulness is practiced in the scanner without neurofeedback, which will allow examination of whether real-time neurofeedback adds significant benefit on top of mindfulness intervention. This is important, as fMRI-based interventions are costly and complex. Third, although all participants reported using mindfulness during the neurofeedback task, it was not assessed for the specificity of mindfulness compared to other strategies or cognitive activities that could also be happening during neurofeedback. Future studies should include a more comprehensive assessment of participants' use of mindfulness during neurofeedback, such as duration, adoption of other strategies, or frequency of switching strategies. Fourth, given the current design, the test does not rule out the possibility that the observed DMN connectivity and SMS changes were at least partially due to changes in participants' anxiety or stress levels pre- vs. post-mbNF. Previous mindfulness-based neurofeedback experiments have shown reduced DMN connectivity using sham-controlled design [A36] as well as SMS increases in the absence of stress level change [A84]. Nevertheless, future studies should include appropriate control conditions as well as assess state anxiety and stress before and after mbNF. Last, the clinical utility of mbNF could be better evaluated with immediate and longitudinal post-mbNF assessment of depression symptoms, particularly as the greatest symptom improvement may happen weeks to months after neurofeedback intervention [A63-A66].

An important future direction in personalizing the mbNF protocol is to determine the optimal session length as well as the number of sessions needed to provide maximal clinical benefit. For example, this study revealed that neurofeedback performance on the last run, not the average, was more predictive of neural and behavioral change, suggesting the length of the mbNF session may vary depending on how quickly a participant reaches a certain threshold of performance. The scalability of the mbNF paradigm will also be greatly improved if this protocol can be implemented in less costly systems such as electroencephalography or functional near-infrared spectroscopy. In the long term, it is the aim to develop a closed-loop system for delivering mbNF intervention when suboptimal brain states (e.g., ruminative, or suicidal) are detected in patients.

In summary, the mbNF protocol is a non-invasive and personalizable tool that may offer early intervention and alleviate depression in adolescents. Building on these promising findings, a key next step is to determine whether this approach leads to improvements in depressive symptoms, which has enormous potential to revolutionize approaches to clinical care.

### REFERENCES

[1] NEIMI. Transforming the understanding and treatment of mental illnesses. https://www.nimh.nih.gov/health/statistics/mental-illness.
[2] Rush, A. J. et al. Acute and longer-term outcomes in depressed outpatients requiring one or several treatment steps: a STAR*D report. Am. J. Psychiatry 163, 1905-1917 (2006).
[3] Wergeland, G. J. H. et al. An effectiveness study of individual vs. group cognitive behavioral therapy for anxiety disorders in youth. Behav. Res. Ther. 57, 1-12 (2014).
[4] Wielgosz, J., Goldberg, S. B., Kral, T. R., Dunne, J. D. & Davidson, R. J. Mindfulness meditation and psychopathology. Annu. Rev. Clin. Psychol. 15, 285-316 (2019).
[5] Oud, M. et al. Effectiveness of CBT for children and adolescents with depression: A systematic review and meta-regression analysis. Eur. Psychiatry 57, 33-45 (2019).
[6] Hamilton, J. P., Farmer, M., Fogelman, P. & Gotlib, I. H. Depressive rumination, the default-mode network, and the dark matter of clinical neuroscience. Biol. Psychiatry 78, 224-230 (2015).
[7] Sheline, Y. I. et al. The default mode network and self-referential processes in depression. Proc. Natl. Acad. Sci. 106, 1942-1947 (2009).
[8] Hampson, M., Driesen, N., Roth, J. K., Gore, J. C. & Constable, R. T. Functional connectivity between task-positive and task-negative brain areas and its relation to working memory performance. Magn. Reson. Imaging 28, 1051-1057 (2010).
[9] Keller, J. B. et al. Resting-state anticorrelations between medial and lateral prefrontal cortex: association with working memory, aging, and individual differences. Cortex 64, 271-280 (2015).
[10] Buckner, R. L. & DiNicola, L. M. The brain's default network: updated anatomy, physiology and evolving insights. Nat. Rev. Neurosci. 20, 593-608 (2019).
[11] Hinds, O. et al. Computing moment-to-moment BOLD activation for real-time neurofeedback. Neuroimage 54, 361-368 (2011).
[12] Yoo, J. J. et al. When the brain is prepared to learn: enhancing human learning using real-time fMRI. Neuroimage 59, 846-852 (2012).
[13] Bauer, C. C. et al. Real-time fMRI neurofeedback reduces auditory hallucinations and modulates resting state connectivity of involved brain regions: Part 2: Default mode network-preliminary evidence. Psychiatry Res. 284, 112770 (2020).
[14] Okano, K. et al. Real-time fMRI feedback impacts brain activation, results in auditory hallucinations reduction: Part 1: Superior temporal gyrus-Preliminary evidence. Psychiatry Res. 286, 112862 (2020).
[15] DeCharms, R. C. et al. Control over brain activation and pain learned by using real-time functional MRI. Proc. Natl. Acad. Sci. 102, 18626-18631 (2005).
[16] Meir-Hasson, Y., Kinreich, S., Podlipsky, I., Hendler, T. & Intrator, N. An EEG Finger-Print of fMRI deep regional activation. Neuroimage 102, 128-141 (2014).
[17] van Lutterveld, R. et al. Source-space EEG neurofeedback links subjective experience with brain activity during effortless awareness meditation. NeuroImage 151, 117-127 (2017).
[18] Ellwood-Lowe M E., Whitfield-Gabrieli S., Bunge S A. (In Press) What is an adaptive pattern of brain network coupling for a child? It depends on their environment. Nature Communications.
[19] Ellwood-Lowe, M. E., Whitfield-Gabrieli, S. & Bunge, S. A. What is an adaptive pattern of brain network coupling for a child? It depends on their environment. Nat. Commun. (in Press).
[20] Whitfield-Gabrieli, S. & Ford, J. M. Default mode network activity and connectivity in psychopathology. Annu. Rev. Clin. Psychol. 8, 49-76 (2012).
[21] Zhang, J. et al. Targeting default mode network connectivity with mindfulness-based fMRI neurofeedback: A pilot study among adolescents with affective disorder history. http://biorxiv.org/lookup/doi/10.1101/2022.08.22.504796 (2022) doi:10.1101/2022.08.22.504796.
[22] Mattfeld, A. T. et al. Brain differences between persistent and remitted attention deficit hyperactivity disorder. Brain 137, 2423-2428 (2014).
[23] Kucyi, A. et al. Prediction of stimulus-independent and task-unrelated thought from functional brain networks. Nat. Commun. 12, 1-17 (2021).
[24] Whitfield-Gabrieli, S. et al. Association of Intrinsic Brain Architecture With Changes in Attentional and Mood Symptoms During Development. JAMA Psychiatry 77, 378-386 (2020).
[25] Whitfield-Gabrieli, S. et al. Understanding marijuana's effects on functional connectivity of the default mode network in patients with schizophrenia and co-occurring cannabis use disorder: A pilot investigation. Schizophr. Res. 194, 70-77 (2018).
[26] Bauer, C. C. et al. Mindfulness training preserves sustained attention and resting state anticorrelation between default-mode network and dorsolateral prefrontal cortex: A randomized controlled trial. Hum. Brain Mapp. (2020).
[27] Bauer, C. C. C., Whitfield-Gabrieli, S., Diaz, J. L., Pasaye, E. H. & Barrios, F. A. From state-to-trait meditation: Reconfiguration of central executive and default mode networks. Eneuro 6, (2019).
[28] Bauer, C. C. et al. Mindfulness training preserves sustained attention and resting state anticorrelation between default-mode network and dorsolateral prefrontal cortex: A randomized controlled trial. Hum. Brain Mapp. (2020).
[29] Posner, J. et al. Dissociable attentional and affective circuits in medication-naïve children with attention-deficit/hyperactivity disorder. Psychiatry Res. Neuroimaging 213, 24-30 (2013).
[30] Sidlauskaite, J., Sonuga-Barke, E., Roeyers, H. & Wiersema, J. R. Altered intrinsic organisation of brain networks implicated in attentional processes in adult attention-deficit/hyperactivity disorder: a resting-state study of attention, default mode and salience network connectivity. Eur. Arch. Psychiatry Clin. Neurosci. 266, 349-357 (2016).
[31] Seli, P., Smallwood, J., Cheyne, J. A. & Smilek, D. On the relation of mind wandering and ADHD symptomatology. Psychon. Bull. Rev. 22, 629-636 (2015).
[32] Lanier, J., Noyes, E. & Biederman, J. Mind wandering (internal distractibility) in ADHD: A literature review. J. Atten. Disord. 25, 885-890 (2021).
[A1] Greenberg P E, Fournier A A, Sisitsky T, Pike C T, Kessler R C, “The economic burden of adults with major depressive disorder in the United States (2005 and 2010);” J. Clin. Psychiatry, 2015; 76; 155-62.
[A2] Avenevoli S, Swendsen J, He J P, Burstein M, Merikangas K R, “Major depression in the national comorbidity survey-adolescent supplement: prevalence, correlates, and treatment;” J. Am. Acad. Child Adolesc. Psychiatry. 2015; 54; 37-44.e2.
[A3] Rush A J, Trivedi M H, Wisniewski S R, Nierenberg A A, Stewart J W, Warden D, et al., “Acute and longer-term outcomes in depressed outpatients requiring one or several treatment steps: a STAR*D report;” Am. J. Psychiatry. 2006; 163; 1905-17.
[A4] Raichle M E, MacLeod A M, Snyder A Z, Powers W J, Gusnard D A, Shulman G L., “A default mode of brain function;” Proc. Natl. Acad. Sci. USA, 2001; 98:676.
[A5] Greicius M D, Krasnow B, Reiss A L, Menon V, “Functional connectivity in the resting brain: a network analysis of the default mode hypothesis;” Proc. Natl. Acad. Sci. USA, 2003; 100; 253-8.
[A6] Kleckner I R, Zhang J, Touroutoglou A, Chanes L, Xia C, Simmons W K, et al., “Evidence for a large-scale brain system supporting allostasis and interoception in humans;” Nat. Hum. Behav. 2017; 1; 0069.
[A7] Margulies D S, Kelly A M C, Uddin L Q, Biswal B B, Castellanos F X, Milham M P, “Mapping the functional connectivity of anterior cingulate cortex;” Neuroimage, 2007; 37; 579-88.
[A8] Greicius M D, Flores B H, Menon V, Glover G H, Solvason H B, Kenna H, et al., “Restingstate functional connectivity in major depression: abnormally increased contributions from subgenual cingulate cortex and thalamus;” Biol. Psychiatry, 2007; 62; 429-37.
[A9] Hamilton J P, Farmer M, Fogelman P, Gotlib I H, “Depressive rumination, the default-mode network, and the dark matter of clinical neuroscience;” Biol. Psychiatry, 2015; 78; 224-30.
[A10] Chai X J, Hirshfeld-Becker D, Biederman J, Uchida M, Doehrmann O, Leonard J A, et al., “Altered intrinsic functional brain architecture in children at familial risk of major depression;” Biol. Psychiatry, 2016; 80; 849-58.
[A11] Michl L C, McLaughlin K A, Shepherd K, Nolen-Hoeksema S., “Rumination as a mechanism linking stressful life events to symptoms of depression and anxiety: longitudinal evidence in early adolescents and adults;” J. Abnorm. Psychol., 2013; 122; 339-52.
[A12] Zhou H-X, Chen X, Shen Y-Q, Li L, Chen N-X, Zhu Z-C, et al., “Rumination and the default mode network: meta-analysis of brain imaging studies and implications for depression;” Neuroimage, 2020; 206; 116287.
[A13] Sheline Y I, Barch D M, Price J L, Rundle M M, Vaishnavi S N, Snyder A Z, et al. “The [00154] default mode network and self-referential processes in depression;” Proc. Natl. Acad. Sci. USA, 2009; 106; 1942-7.
[A14] Rayner G, Jackson G, Wilson S., “Cognition-related brain networks underpin the symptoms of unipolar depression: evidence from a systematic review;” Neurosci. Biobehav. Rev., 2016; 61; 53-65.
[A15] Fossati P., “Circuit based anti-correlation, attention orienting, and major depression;” CNS Spectr., 2019; 24; 94-101.
[A16] Abela J R Z, Hankin B L., “Rumination as a vulnerability factor to depression during the transition from early to middle adolescence: a multiwave longitudinal study;” J. Abnorm. Psychol., 2011; 120; 259-71.
[A17] Michalak J, Holz A, Teismann T., “Rumination as a predictor of relapse in mindfulness-based cognitive therapy for depression;” Psychol. Psychother., 2011; 84; 230-6.
[A18] Grassia M, Gibb B E., “Rumination and prospective changes in depressive symptoms;” J. Soc. Clin. Psychol., 2008; 27; 931-48.
[A19] Jones N P, Siegle G J, Thase M E., “Effects of rumination and initial severity on remission to cognitive therapy for depression;” Cognit. Ther. Res., 2008; 32; 591-604.
[A20] Whitfield-Gabrieli S, Ford J M., “Default mode network activity and connectivity in psychopathology;” Annu. Rev. Clin. Psychol., 2012; 8; 49-76.
[A21] Liston C, Chen A C, Zebley B D, Drysdale A T, Gordon R, Leuchter B, et al., “Default mode network mechanisms of transcranial magnetic stimulation in depression;” Biol. Psychiatry, 2014; 76; 517-26.
[A22] Brewer J A, Worhunsky P D, Gray J R, Tang Y-Y, Weber J, Kober H., “Meditation experience is associated with differences in default mode network activity and connectivity;” Proc. Natl. Acad. Sci. USA, 2011; 108; 20254-9.
[A23] Ives-Deliperi V L, Solms M, Meintjes E M., “The neural substrates of mindfulness: an fMRI investigation;” Soc. Neurosci., 2011; 6; 231-42.
[A24] Feruglio S, Matiz A, Pagnoni G, Fabbro F, Crescentini C., “The impact of mindfulness meditation on the wandering mind: a systematic review;” Neurosci. Biobehav. Rev., 2021; 131; 313-30.
[A25] Scheibner H J, Bogler C, Gleich T, Haynes J-D, Bermpohl F., “Internal and external attention and the default mode network;” Neuroimage, 2017; 148; 381-9.
[A26] Hasenkamp W, Wilson-Mendenhall C D, Duncan E, Barsalou L W., “Mind wandering and attention during focused meditation: a fine-grained temporal analysis of fluctuating cognitive states;” Neuroimage, 2012; 59; 750-60.
[A27] Bauer C C C, Whitfield-Gabrieli S, Diaz J L, Pasaye E H, Barrios F A., “From state-to-trait meditation: reconfiguration of central executive and default mode networks;” eNeuro., 2019; 6; 6.
[A28] Hofmann S G, Gómez A F., “Mindfulness-based interventions for anxiety and depression;” Psychiatr. Clin. North. Am., 2017; 40; 739-49.
[A29] Strohmaier S, Jones F W, Cane J E., “Effects of length of mindfulness practice on mindfulness, depression, anxiety, and stress: a randomized controlled experiment;” Mindfulnes, 2021; 12: 198-214.
[A30] Khoury B, Lecomte T, Fortin G, Masse M, Therien P, Bouchard V, et al., “Mindfulness-based therapy: a comprehensive meta-analysis;” Clin. Psychol. Rev., 2013; 33; 763-71.
[A31] Wielgosz J, Goldberg S B, Kral T R A, Dunne J D, Davidson R J., “Mindfulness meditation and psychopathology;” Annu. Rev. Clin. Psychol., 2019; 15; 285-316.
[A32] Bauer C C C, Caballero C, Scherer E, West M R, Mrazek M D, Phillips D T, et al., “Mindfulness training reduces stress and amygdala reactivity to fearful faces in middle-school children;” Behav. Neurosci., 2019; 133; 569-85.
[A33] Bauer C C C, Rozenkrantz L, Caballero C, Nieto-Castanon A, Scherer E, West M R, et al., “Mindfulness training preserves sustained attention and resting state anticorrelation between default-mode network and dorsolateral prefrontal cortex: a randomized controlled trial;” Hum. Brain Mapp., 2020, https://doi.org/10.1002/hbm.25197.
[A34] Bauer C C C, Zhang J, Morfini F, Kucyi A, Raya J, Urban Z, et al., “REMind: real-time neurofeedback enhanced mindfulness protocol using multivariate and univariate real-time functional imaging (MURFI);” 2022.
[A35] Tursic A, Eck J, Luhrs M, Linden D E J, Goebel R., “A systematic review of fMRI neurofeedback reporting and effects in clinical populations;” Neuroimage Clin., 2020; 28; 102496.
[A36] Bauer C C C, Okano K, Ghosh S S, Lee Y J, Melero H, Angeles C, et al., “Real-time fMRI neurofeedback reduces auditory hallucinations and modulates resting state connectivity of involved brain regions: part 2: default mode network-preliminary evidence;” Psychiatry Res., 2020; 284; 112770.
[A37] Okano K, Bauer C C C, Ghosh S S, Lee Y J, Melero H, de Los Angeles C, et al., “Realtime fMRI feedback impacts brain activation, results in auditory hallucinations reduction: part 1: superior temporal gyrus-preliminary evidence;” Psychiatry Res., 2020; 286; 112862.
[A38] Hubbard N A, Siless V, Frosch I R, Goncalves M, Lo N, Wang J, et al., “Brain function and clinical characterization in the Boston adolescent neuroimaging of depression and anxiety study;” Neuroimage Clin., 2020; 27; 102240.
[A39] Siless V, Hubbard N A, Jones R, Wang J, Lo N, Bauer C C C, et al., “Image acquisition and quality assurance in the Boston adolescent neuroimaging of depression and anxiety study;” Neuroimage Clin., 2020; 26; 102242.
[A40] Kaufman J, Birmaher B, Brent D, Rao U, Flynn C, Moreci P, et al., “Schedule for affective disorders and schizophrenia for school-age children-present and lifetime version (K-SADS-PL): initial reliability and validity data;” J. Am. Acad. Child Adolesc. Psychiatry, 1997; 36; 980-8.
[A41] Angold A, Costello E J, Messer S C, Pickles A., “Development of a short questionnaire for use in epidemiological studies of depression in children and adolescents;” Int. J. Methods Psychiatr. Res., 1995; 5; 237-49.
[A42] de Ross R L, Gullone E, Chorpita B F., “The revised child anxiety and depression scale: a psychometric investigation with Australian youth;” Behav. Change, 2002; 19; 90-101.
[A43] Jenkinson M, Beckmann C F, Behrens T E J, Woolrich M W, Smith S M. F S L., Neuroimage, 2012; 62; 782-90.
[A44] Franco A R, Pritchard A, Calhoun V D, Mayer A R., “Interrater and intermethod reliability of default mode network selection;” Hum. Brain Mapp., 2009; 30; 2293-303.
[A45] Tanay G, Bernstein A., “State mindfulness scale (SMS): development and initial validation;” Psychol. Assess., 2013; 25; 1286-99.
[A46] Sayadaw C., “Practical Insight Meditation;” Yangon, Myanmar: Chanmyay Yeiktha Meditation Centre; 2017.
[A47] Peirce J W, “PsychoPy-psychophysics software in Python;” J. Neurosci. Methods, 2007; 162; 8-13.
[A48] Hinds O, Ghosh S, Thompson T W, Yoo J J, Whitfield-Gabrieli S, Triantafyllou C, et al., “Computing moment-to-moment BOLD activation for real-time neurofeedback;” Neuroimage, 2011; 54; 361-8.
[A49] Chen A C, Oathes D J, Chang C, Bradley T, Zhou Z-W, Williams L M, et al., “Causal interactions between fronto-parietal central executive and default-mode networks in humans;” Proc. Natl. Acad. Sci. USA, 2013; 110; 19944-9.
[A50] Esteban O, Markiewicz C J, Blair R W, Moodie C A, Isik A I, Erramuzpe A, et al., “fMRIPrep: a robust preprocessing pipeline for functional MRI;” Nat. Methods, 2019; 16; 111-6.
[A51] Gorgolewski K, Burns C D, Madison C, Clark D, Halchenko Y O, Waskom M L, et al., “Nipype: a flexible, lightweight and extensible neuroimaging data processing framework in python;” Front Neuroinform., 2011; 5; 13.
[A52] Whitfield-Gabrieli S, Nieto-Castanon A., “Conn: a functional connectivity toolbox for correlated and anticorrelated brain networks;” Brain Connect., 2012; 2; 125-41.
[A53] Nieto-Castanon A., “Handbook of functional connectivity magnetic resonance imaging methods in CONN;” Hilbert Press; 2020, https://doi.org/10.56441/hilbertpress.2207.6598.
[A54] Behzadi Y, Restom K, Liau J, Liu T T., “A component based noise correction method (CompCor) for BOLD and perfusion based fMRI;” Neuroimage, 2007; 37; 90-101.
[A55] Taxali A, Angstadt M, Rutherford S, Sripada C., “Boost in test-retest reliability in resting state fMRI with predictive modeling;” Cereb Cortex, 2021; 31; 2822-33.
[A56] Caceres A, Hall D L, Zelaya F O, Williams S C R, Mehta M A, “Measuring fMRI reliability with the intra-class correlation coefficient;” Neuroimage, 2009; 45; 758-68.
[A57] Whitfield-Gabrieli S, Wendelken C, Nieto-Castañón A, Bailey S K, Anteraper S A, Lee Y J, et al., “Association of intrinsic brain architecture with changes in attentional and mood symptoms during development;” JAMA Psychiatry, 2020; 77; 378-86.
[A58] Jenkinson M, Bannister P, Brady M, Smith S., “Improved optimization for the robust and accurate linear registration and motion correction of brain images;” Neuroimage, 2002; 17; 825-41.
[A59] Fox M D, Snyder A Z, Vincent J L, Corbetta M, Van Essen D C, Raichle M E, “The human brain is intrinsically organized into dynamic, anticorrelated functional networks;” Proc. Natl. Acad. Sci. USA, 2005; 102; 9673-8.
[A60] Baron R M, Kenny D A, “The moderator-mediator variable distinction in social psychological research: conceptual, strategic, and statistical considerations;” J. Pers. Soc. Psychol., 1986; 51; 1173-82.
[A61] Preacher K J, Hayes A F, “Asymptotic and resampling strategies for assessing and comparing indirect effects in multiple mediator models;” Behav. Res. Methods, 2008; 40; 879-91.
[A62] Garrison K A, Zeffiro T A, Scheinost D, Constable R T, Brewer J A, “Meditation leads to reduced default mode network activity beyond an active task;” Cogn. Affect. Behav. Neurosci., 2015; 15; 712-20.
[A63] Rance M, Walsh C, Sukhodolsky D G, Pittman B, Qiu M, Kichuk S A, et al., “Time course of clinical change following neurofeedback;” Neuroimage, 2018; 181; 807-13.
[A64] Amano K, Shibata K, Kawato M, Sasaki Y, Watanabe T., “Learning to associate orientation with color in early visual areas by associative decoded fMRI neurofeedback;” Curr. Biol., 2016; 26; 1861-6.
[A65] Robineau F, Meskaldji D E, Koush Y, Rieger S W, Mermoud C, Morgenthaler S, et al., “Maintenance of voluntary self-regulation learned through real-time fMRI neurofeedback;” Front. Hum. Neurosci., 2017; 11; 131.
[A66] Megumi F, Yamashita A, Kawato M, Imamizu H., “Functional MRI neurofeedback training on connectivity between two regions induces long-lasting changes in intrinsic functional network. Front Hum Neurosci. 2015; 9:160.”
[A67] Öngür D, Drevets W C, Price J L., “Glial reduction in the subgenual prefrontal cortex in mood disorders;” Proc. Natl. Acad. Sci. USA, 1998; 95; 13290-5
[A68] Drevets W C, Price J L, Simpson J R Jr, Todd R D, Reich T, Vannier M, et al., “Subgenual prefrontal cortex abnormalities in mood disorders;” Nature, 1997; 386; 824-7.
[A69] Mayberg H S, Liotti M, Brannan S K, McGinnis S, Mahurin R K, Jerabek P A, et al., “Reciprocal limbic-cortical function and negative mood: converging PET findings in depression and normal sadness;” Am. J. Psychiatry, 1999; 156; 675-82.
[A70] Ducharme S, Albaugh M D, Hudziak J J, Botteron K N, Nguyen T-V, Truong C, et al., “Anxious/depressed symptoms are linked to right ventromedial prefrontal cortical thickness maturation in healthy children and young adults;” Cereb Cortex. 2014; 24; 2941-50.
[A71] Auerbach R P, Pagliaccio D, Hubbard N A, Frosch I, Kremens R, Cosby E, et al., “Reward-related neural circuitry in depressed and anxious adolescents: a Human Connectome Project;” J. Am. Acad. Child Adolesc. Psychiatry, 2021; 61; 308-20.
[A72] Rodriguez-Cano E, Sarró S, Monte G C, Maristany T, Salvador R, McKenna P J, et al., “Evidence for structural and functional abnormality in the subgenual anterior cingulate cortex in major depressive disorder;” Psychol. Med., 2014; 44; 3263-73.
[A73] LeWinn K Z, Connolly C G, Wu J, Drahos M, Hoeft F, Ho T C, et al., “White matter correlates of adolescent depression: structural evidence for frontolimbic dysconnectivity;” J. Am. Acad. Child Adolesc. Psychiatry, 2014; 53; 899-909.
[A74] Heij G J, Penninx B W H J, van Velzen L S, van Tol M-J, van der Wee N J A, Veltman D J, et al., “White matter architecture in major depression with anxious distress symptoms;” Prog. Neuropsychopharmacol. Biol. Psychiatry, 2019; 94; 109664.
[A75] Bracht T, Linden D, Keedwell P., “A review of white matter microstructure alterations of pathways of the reward circuit in depression;” J. Affect. Disord., 2015; 187; 45-53.
[A76] Barrett L F, Quigley K S, Hamilton P., “An active inference theory of allostasis and interoception in depression;” Philos. Trans. R. Soc. Lond. B. Biol. Sci., 2016; 371; 1708.
[A77] Mayberg H S, Lozano A M, Voon V, McNeely H E, Seminowicz D, Hamani C, et al., “Deep brain stimulation for treatment-resistant depression;” Neuron, 2005; 45; 651-60.
[A78] Riva-Posse P, Choi K S, Holtzheimer P E, Crowell A L, Garlow S J, Rajendra J K, et al., “A connectomic approach for subcallosal cingulate deep brain stimulation surgery: prospective targeting in treatment-resistant depression;” Mol. Psychiatry, 2018; 23; 843-9.
[A79] Fox M D, Buckner R L, White M P, Greicius M D, Pascual-Leone A., “Efficacy of transcranial magnetic stimulation targets for depression is related to intrinsic functional connectivity with the subgenual cingulate;” Biol. Psychiatry, 2012; 72; 595-603.
[A80] Cash R F H, Zalesky A, Thomson R H, Tian Y, Cocchi L, Fitzgerald P B, “Subgenual functional connectivity predicts antidepressant treatment response to transcranial magnetic stimulation: independent validation and evaluation of personalization; Biol, Psychiatry, 2019; 86; e5-7.
[A81] Weigand A, Horn A, Caballero R, Cooke D, Stern A P, Taylor S F, et al., “Prospective validation that subgenual connectivity predicts antidepressant efficacy of transcranial magnetic stimulation sites;” Biol. Psychiatry, 2018; 84; 28-37.
[A82] Harrison R, Zeidan F, Kitsaras G, Ozcelik D, Salomons T V, “Trait mindfulness is associated with lower pain reactivity and connectivity of the default mode network;” J. Pain, 2019; 20; 645-54.
[A83] Hunt C, Letzen J E, Krimmel S R, Burrowes S A B, Haythornthwaite J A, Finan P, et al., “Is mindfulness associated with lower pain reactivity and connectivity of the default mode network? A replication and extension study in healthy and episodic migraine participants;” J. Pain, 2022; 23; 2110-20.
[A84] Kirlic N, Cohen Z P, Tsuchiyagaito A, Misaki M, McDermott T J, Aupperle R L, et al., “Self-regulation of the posterior cingulate cortex with real-time fMRI neurofeedback augmented mindfulness training in healthy adolescents: a nonrandomized feasibility study;” Cogn. Affect. Behav. Neurosci., 2022; 22; 849-67.
[A85] Ramot M, Gonzalez-Castillo J., “A framework for offline evaluation and optimization of real-time algorithms for use in neurofeedback, demonstrated on an instantaneous proxy for correlations;” Neuroimage, 2019; 188; 322-34.
[A86] Zhang J, Kucyi A, Raya J, Nielsen A N, Nomi J S, Damoiseaux J S, et al., “What have we really learned from functional connectivity in clinical populations?” Neuroimage, 2021; 242; 118466.
[A87] Insel T R., “The NIMH Research Domain Criteria (RDoC) Project: precision medicine for psychiatry;” Am. J. Psychiatry, 2014; 171; 395-7.
[A88] McClintock S M, Reti I M, Carpenter L L, McDonald W M, Dubin M, Taylor S F, et al., “Consensus recommendations for the clinical application of repetitive transcranial magnetic stimulation (rTMS) in the treatment of depression;” J. Clin. Psychiatry, 2018; 79; 16cs10905.
[A89] Mayberg H S, Riva-Posse P, Crowell A L., “Deep brain stimulation for depression: keeping an eye on a moving target;” JAMA Psychiatry., 2016; 73; 439-40.
[A90] Sorger B, Scharnowski F, Linden D E J, Hampson M, Young K D. Control freaks: towards optimal selection of control conditions for fMRI neurofeedback studies. Neuroimage. 2019; 186:256-65.

The teachings of all patents, published applications, and references cited herein or in the manuscript being filed herewith are incorporated by reference in their entirety.

While example embodiments have been particularly shown and described, it will be understood by those skilled in the art that various changes in form and details may be made therein without departing from the scope of the embodiments encompassed or contemplated herein or in the manuscript being filed herewith.

What is claimed is:

1. A neurofeedback method for modulating brain activity, the method comprising:
functionally locating at least two brain networks in an adolescent subject during a resting state, at least one of the brain networks being associated with affective disorder symptoms during adolescence;
recording signals from each of the brain networks while the subject is performing a meditation task;
determining an activity metric based on a difference derived from the recorded signals, wherein changes in the activity metric over time are indicative of changes in relative activity level of the brain networks; and
providing neurofeedback by delivering a representation of the activity metric to the subject during the recording, thereby modulating brain activity in the subject.

2. The method of claim 1, wherein the functionally locating comprises using functional magnetic resonance imaging to generate a network map for each of the brain networks.

3. The method of claim 1, wherein the signals are blood-oxygen-level-dependent (BOLD) signals recorded using real-time functional magnetic resonance imaging (rt-fMRI).

4. The method of claim 1, wherein the signals are electrical signals recorded using fMRI compatible simultaneous electroencephalography.

5. The method of claim 1, wherein modulating the brain activity comprises downregulating or decreasing activity level in one of the brain networks.

6. The method of claim 1, wherein modulating the brain activity comprises upregulating or increasing activity level in one of the brain networks.

7. The method of claim 1, wherein the meditation task comprises mindfulness meditation.

8. The method of claim 1, wherein the brain networks include, a default mode network (DMN), a central executive network (CEN), or a frontoparietal control network (FPCN).

9. The method of claim 1, further comprising determining a first resting state activity before the performing of the meditation task, determining a second resting state activity after the performing of the meditation task, and assessing a change in functional connectivity of at least one of the brain networks based on a comparison between the first and second resting state activities.

10. The method of claim 1, wherein the subject is suffering from a major depressive disorder.

11. A neurofeedback system for modulating brain activity, the system comprising:
a processor;
a sensor; and
a memory with computer code instructions stored thereon, the processor, the sensor, and the memory, being configured to cause the system to:
functionally locate at least two brain networks in an adolescent subject during a resting state, the at least one brain networks being associated with affective disorder symptoms during adolescence;
record signals from each of the brain networks while the subject is performing a meditation task;
determine an activity metric based on a difference derived from the recorded signals, wherein changes in the activity metric over time are indicative of changes in relative activity level of the brain networks; and
provide neurofeedback to the subject by delivering a representation of the activity metric to the subject during the recording, thereby modulating brain activity in the subject.

12. The system of claim 11, wherein the functionally locating comprises using frequency specific components of electroencephalography signals as correlates of functional magnetic resonance imaging to generate a network map for each of the brain networks.

13. The system of claim 12, wherein the electroencephalography signals are recorded on the scalp of the user wearing the system.

14. The system of claim 12, wherein using frequency specific components of the electroencephalography signals as correlates of functional magnetic resonance imaging further includes using user specific electroencephalography signals processed with user specific functional magnetic resonance imaging data to generate central executive network (CEN) dynamics, default mode network (DMN) dynamics, or both.

15. The system of claim 11, wherein the modulating comprises downregulating or decreasing activity level in one of the brain networks.

16. The system of claim 11, wherein the modulating comprises upregulating or increasing activity level in one of the brain networks.

17. The system of claim 11, wherein the meditation task comprises mindfulness meditation.

18. The system of claim 11, wherein the brain networks include a default mode network (DMN), a central executive network (CEN), or a frontoparietal control network (FPCN).

19. The system of claim 11, wherein the system is further configured to determine a first resting state activity level before the performing of the meditation task, determine a second resting state activity level after the performing of the meditation task, and assess a change in functional connectivity of at least one of the brain networks based on a comparison between the first and second resting state activity levels.

20. The system of claim 11, wherein the neurofeedback provided to the subject is visually provided on a screen.
